(19) United States
(12) Patent Application Publication
(10) Pub. No.: US 2020/0155642 A1
(43) Pub. Date: May 21, 2020

(54) PURIFICATION OF ALGAL EXTRACTS AND THEIR APPLICATIONS

(71) Applicant: Back of the Yards Algae Sciences LLC, Chicago, IL (US)

(72) Inventor: Leonard Lerer, Chicago, IL (US)

(21) Appl. No.: 16/685,363

(22) Filed: Nov. 15, 2019

Related U.S. Application Data
(60) Provisional application No. 62/768,876, filed on Nov. 17, 2018.
(60) Provisional application No. 62/808,976, filed on Feb. 22, 2019.

(51) Int. Cl. A61K 38/16; A61K 35/748

(52) U.S. Cl. CPC A61K 38/164; A61K 35/748

(57) ABSTRACT

The present invention relates to the field of purification of algal extracts, methods for their preparation, the unique composition of such purified extracts and their applications. A particular, detailed example of the invention is provided relating to phycobiliprotein compositions and methods for their preparation and in particular to phycobiliprotein compositions with unique molecular markers. The phycobiliprotein compositions are characterized by unique protein components as assayed by 2-D gel electrophoresis. The present invention encompasses oral, topical, mucosal, nasal, ophthalmological and other formulations containing the phycobiliprotein compositions, either alone or in combination with other active agents. The unique compositions may be used for a large range of applications including to treat a variety of diseases and conditions, as a food and beverage ingredient, nutraceutical ingredient and food colorant, and in agriculture, animal nutrition, bioremediation and cell culture. The invention can be used for the purification of extracts from a range of algae.

### CROSS-REFERENCE TO RELATED APPLICATIONS

This application claims the benefit of U.S. Prov. Appl. 62/768,876, filed Nov. 17, 2018, and U.S. Prov. Appl. 62/808,976, filed Feb. 22, 2018, each of which is incorporated herein by reference in its entirety.

### FIELD OF THE INVENTION

The present invention relates to the field of purification of algal extracts, methods for their preparation, the unique composition of such purified extracts and their applications

### BACKGROUND OF THE INVENTION

Algae are a diverse group of organisms that include macroalgae (also called ‘seaweed’) and microalgae. Microalgae are microscopic algae and include cyanobacteria (also called ‘blue-green algae’). Microalgae are typically found in freshwater and marine systems and have various shapes with a diameter or length of approximately 3-10 μm. The term microalgae includes prokaryotic and eukaryotic organisms. Microalgae, capable of performing photosynthesis, are important for life on earth as they produce approximately half of all the atmospheric oxygen. Microalgae are a source of a wide range of substances and extracts with applications human and animal nutrition, foods and beverages, human and animal health, medicine and pharmaceuticals, wellness and nutraceuticals and functional foods, agriculture including organic, biodynamic, general, aquaponics, regenerative, cellular and other types of agriculture, cell culture, plant propagation and soil amendment and pollution control and bioremediation and other possible applications. Spirulina (generally, Arthrospira platensis, a blue-green microalgae also known as a cyanobacteria) is the best-known microalgae and has been used as a food, for hundreds of years, in Africa, Asia and South America. Spirulina is currently consumed, all over the world, as a health food and a phycobiliprotein-rich extract of Spirulina (generally called phycocyanin) is currently used as a natural blue food colorant, and as a nutraceutical and laboratory reagent, although it has many other potential applications. Phycobiliproteins are water-soluble proteins that play a key role in photosynthesis.

Phycobiliproteins take the form of a complex between proteins and covalently bound phycobilins that act as chromophores. They are the most important constituents of the phycobilisome. The major phycobiliproteins are C- phycocyanin, R-phycoerythrin, B-phycoerythrin and allophycocyanin. The blue phycobiliprotein (PBP) pigment complex contained in Spirulina that is generally called phycocyanin (PC) plays an important role in the photosynthetic chain acting as the link between light energy and chlorophyll. A number of different algae including Porphyridium cruentum, Galdieria sulphuria and Aphanizomenon flos aquae (AFA), other cyanobacteria and other organisms contain varying amounts of PBP complexes.

There is a growing demand for PBP pigments, mainly PC, especially as a natural food colorant to replace chemical food dyes. PC is mainly produced through a process of extraction, purification and drying from Spirulina and a number of methods have been developed to extract and purify PC. Such methods would be applicable to other cyanobacteria and PCB containing organisms.

The first step in producing PC is extraction whereby the cell wall of Spirulina is weakened or broken in order to liberate the contents of the cell that includes mainly chlorophyll and up to 25% PC depending the genetic structure and method of cultivation of the Spirulina. Extraction through cell wall destruction can be achieved by physical methods (ultrasound, high pressure, ball mills, freeze-thaw cycles), by chemical methods (strong acids, strong alkalis or calcium ions), by enzymatic processes (lysozyme treatment) or through a combination of these methods. After extraction, the PC and other cell constituents exist in an aqueous solution mixed with cellular debris and insoluble or fat-soluble substances. PC accompanied by other water-soluble substances can be separated from the cellular debris and insoluble substances by methods such as centrifugation or tangential filtration (as taught in the document FR2789399). Solvents other than water can be used for the extraction, such as glycerol or water-glycerol mixtures (see WO2014045177).

The document CN1273795 teaches breaking Spirulina cell walls by the addition of a large quantity of a solubilizing salt (sodium chloride, sodium acetate, calcium chloride or other), permits one to obtain, in a 2-3 day period, a supernatant rich in PC; the process includes a necessary step of separation by filtration or centrifugation followed by dialysis to remove the remaining salts.

### SUMMARY OF THE INVENTION

The present invention relates to the field of purification of algal extracts, methods for their preparation, the unique composition of such purified extracts and their applications. The primary example of this invention is the preparation through purification of a purified PC-rich extract of Spirulina (PPES), and in particular the PBP compositions of this purified extract with unique molecular markers. A second example of this invention's method of preparation in the field of purification of algal extracts is the preparation of Chlorella extract, also known as Chlorella Growth Factor (CGF). This invention's method can be applied to many different cyanobacteria and PC containing organisms.

In some embodiments, the present invention provides a purified PBP composition characterized by one or more of the following characteristics: the a) the protein fraction of the composition comprises greater than about 30% of a protein having a molecular weight of about 17,695 kDa and an isoelectric point of about 6.29 as assayed by two dimensional gel electrophoresis followed by quantitation by densitometry; b) the protein fraction of the composition comprises greater than about 5% of a protein having a molecular weight of about 19,833 kDa and an isoelectric point of about 6.14 as assayed by two dimensional gel electrophoresis followed by quantitation by densitometry; c) the protein fraction of the composition comprises greater than about 1% of a protein having a molecular weight of about 45,578 kDa and an isoelectric point of about 6.2 as assayed by two dimensional gel electrophoresis followed by quantitation by densitometry; d) the protein fraction of the composition comprises greater than about 1% of a protein having a molecular weight of about 35,014 kDa and an isoelectric point of about 5.9 as assayed by two dimensional gel electrophoresis followed by quantitation by densitometry; e) the protein fraction of the composition comprises greater than about 0.30% of a protein having a molecular weight of about 24,688 kDa and an isoelectric point of about 5.3 as assayed by two dimensional gel electrophoresis followed by quantitation by densitometry; f) the protein fraction of the composition comprises greater than about 2% of a protein having a molecular weight of about 22,522 kDa and an isoelectric point of about 5.9 as assayed by two dimensional gel electrophoresis followed by quantitation by densitometry; g) the protein fraction of the composition comprises greater than about 1% of a protein having a molecular weight of about 21,023 kDa and an isoelectric point of about 7.3 as assayed by two dimensional gel electrophoresis followed by quantitation by densitometry; h) the protein fraction of the composition comprises greater than about 0.50% of a protein having a molecular weight of about 13,417 kDa and an isoelectric point of about 7.3 as assayed by two dimensional gel electrophoresis followed by quantitation by densitometry; i) the protein fraction of the composition comprises a ratio of major protein constituents to minor protein constituents of less than 3.5:1 based on the aggregate mass of the proteins, wherein major protein constituents are the protein having a molecular weight of about 17,695 kDa and an isoelectric point of about 6.29 as assayed by two dimensional gel electrophoresis followed by quantitation by densitometry and the protein having a molecular weight of about 19,833 kDa and an isoelectric point of about 6.14 as assayed by two dimensional gel electrophoresis followed by quantitation by densitometry and the minor protein constituents are the remainder of the proteins; j) the protein fraction of the composition comprises less than about 75% on a mass basis of the combined amounts of the protein having a molecular weight of about 17,695 kDa and an isoelectric point of about 6.29 as assayed by two dimensional gel electrophoresis followed by quantitation by densitometry and the protein having a molecular weight of about 19,833 kDa and an isoelectric point of about 6.14 as assayed by two dimensional gel electrophoresis followed by quantitation by densitometry; and k) the composition produces a solution having a color value (based on absorbance) of greater than 200 E 10%/1 cm when 250 mg of a dry powder of the composition are dissolved in one liter of water and absorbance is measured at 618 nm.

In some preferred embodiments, the composition has at least two of characteristics a, b, c, d, e, f, g, h, i, j and k. In some preferred embodiments, the composition has at least three of characteristics a, b, c, d, e, f, g, h, i, j and k. In some preferred embodiments, the composition has at least four of characteristics a, b, c, d, e, f, g, h, i, j and k. In some preferred embodiments, the composition has at least five of characteristics a, b, c, d, e, f, g, h, i, j and k. In some preferred embodiments, the composition has at least six of characteristics a, b, c, d, e, f, g, h, i, j and k. In some preferred embodiments, the composition has at least seven of characteristics a, b, c, d, e, f, g, h, i, j and k. In some preferred embodiments, the composition has at least eight of characteristics a, b, c, d, e, f, g, h, i, j and k. In some preferred embodiments, the composition has at least nine of characteristics a, b, c, d, e, f, g, h, i, j and k. In some preferred embodiments, the composition has at least ten of characteristics a, b, c, d, e, f, g, h, i, j and k. In some preferred embodiments, the composition has all eleven characteristics a, b, c, d, e, f, g, h, i, j and k.

In some preferred embodiments, the purified PBP composition is produced by a process comprising: mixing PBP containing organism biomass with water and gelling agent, forming a droplet, introducing a droplet of the first solution into a second solution containing a salt of divalent cations under conditions such that microcapsules form, and obtaining a purified extract rich in PBP by mixing the microcapsules with a volume of an aqueous solution under conditions such that the PBP diffuses from the microcapsules into the aqueous solution.

In some preferred embodiments, the purified PBP composition is produced by a process comprising: mixing PBP containing organism biomass with water and a salt of divalent cations, forming a droplet, introducing a droplet of the first solution into a second solution containing a gelling agent under conditions that microcapsules form, and obtaining an extract enriched for PBP by mixing the microcapsules with a volume of an aqueous solution under conditions such that the PBP diffuses from the microcapsules into the aqueous solution.

In some preferred embodiments, the purified PBP composition is produced by a process comprising: mixing PBP containing organism biomass with water to form the first solution and coating a droplet, extruded tube, sausage or some other form of this first solution with a second solution of gelling agent and introducing the coated droplet, extruded tube, sausage or some other form of the first solution into a third solution containing a salt of divalent cations under conditions such that a droplet, extruded tube sausage or some other form is obtained, and thereafter obtaining purified extract rich in PBP by mixing the droplets, extruded tubes, sausages or some other forms with a volume of an aqueous solution under conditions such that the PBP diffuses from the droplet, extruded tube, sausage or some other form into the aqueous solution.

In some preferred embodiments, the different methods previously described to produce the purified PBP compositions can be used simultaneously, sequentially or repeatedly.

In some preferred embodiments, the PBP containing organism biomass is specially prepared through heat, cold, chemical and biological processes and physical mechanisms and techniques to facilitate the purification process of this invention.

In some preferred embodiments, the PBP composition comprises a dried powder comprising a purified PBP composition as described above, the powder having a residual moisture content of less than about 10% w/w of the powder.

In some preferred embodiments, the PBP composition comprises fresh or freshly harvested PBP containing organism biomass.

In some preferred embodiments, the microcapsule or other form contains, or is coated with, chemicals or biological agents that enhance or retard or selectively control the diffusion of the PBP.

In some preferred embodiments, the microcapsule or other form contains a natural substance that is purified or concentrated in conjunction with or ‘chaperoned’ into the aqueous solution by the PBP or some other substance that exists in the organism biomass.

In some preferred embodiments the aqueous solution into which the PBP diffuses is modified by changing its temperature, through agitation or mixing or the addition of chemical or biological agents such as acids, alkalis, salts and antimicrobial agents to enhance or retard diffusion of PBP and to prevent contamination.

In some preferred embodiments, the microcapsule or other form is frozen, dried or treated in some other way to enhance or retard diffusion of the PBP into the aqueous solution

In some preferred embodiments, the compositions further comprise a second biologically active agent. In some preferred embodiments, the second biologically active agent is a pharmaceutical agent. In some preferred embodiments, the second biologically active agent is a nutraceutical agent. In some preferred embodiments, the second biologically active agent is an agricultural agent. In some preferred embodiments, the second biologically active agent is a cellular culture agent for plant, meat, poultry, porcine, bovine or other animal or plant or other organism cell culture.

In some preferred embodiments, the present invention provides an oral delivery vehicle comprising a PBP composition as described above. In some preferred embodiments, the oral delivery vehicle is selected from the group consisting of a capsule, a tablet and a gummi gel.

In some preferred embodiments, the present invention provides a delivery vehicle for agricultural applications including as a biostimulant and other applications such as bioremediation and soil amendment. In some preferred embodiments the delivery vehicle is a matrix or framework together with natural and artificial matrices and frameworks including mycelia, fungal carpets, combinations of bacteria and other natural substances including ‘sympathetic combinations of bacteria and yeasts’, matrices and frameworks based on nanotechnology, naturally existing plant matrices, collagen, bone and other tissue matrices and other natural or artificial materials to which the PBP composition can be attached, stuck, incorporated, bound or combined.

In some preferred embodiments, the present invention provides a formulation comprising a PBP composition as described above and a physiologically or pharmaceutically acceptable carrier. In some preferred embodiments, the formulation is selected from a topical formulation, an oral formulation, a mucosal formulation, an ophthalmological formulation and an intranasal formulation.

In some preferred embodiments, the PBP compositions comprise an antioxidant or stabilization agents. Preferred agents may selected from food and/or pharmaceutical grade glycerol, polyethylene glycol, glycine, serum albumin, acacia gum, agar, align, alginate and salts of alginate, carrageenan and salts of carrageenan, furcellaran, arabino-galactan, glycan, calcium carbonate, citrate and salts of citrate, gluconate, calcium phosphate, calcium sulphate, calcium tartrate, carboxymethyl cellulose and salts of CMC, cellulose gum, gelatin, gellan gum, guar gum, gum Arabic, lecithin, hydroxypropyl cellulose, hydroxypropyl methylcellulose, karaya gum, locust bean gum, magnesium chloride, methylcellulose, oat gum, pectin, ester of fatty acids, mono- and diglycerides, tragacanth gum, and xanthan gum.

In some preferred embodiments, the present invention provides a food product comprising a PBP composition as described above and an additional food ingredient that does not naturally occur in Spirulina.

In some preferred embodiments, the spent microcapsules or other forms from which the PBP has diffused into the aqueous solution is a food product, food ingredient, animal feed, nutraceutical, food texturizer, pet food ingredient, fish food, soil amendment, fertilizer, agricultural biostimulant, bioremediation substance or some other product of value.

In some preferred embodiments, the present invention provides a beverage product comprising a PBP composition as described above in an aqueous solution and an additional beverage ingredient that does not naturally occur in Spirulina.

In some preferred embodiments, the present invention provides processes for preparing an aqueous extract rich in PBP from PBP containing microorganisms comprising: mixing a PBP containing microorganism biomass with water and gelling agent, forming microcapsule by introducing the first solution into a second solution containing a salt of divalent cations under conditions such that microcapsules form, and obtaining an extract enriched for PBP by mixing the microcapsules with a volume of an aqueous solution under conditions such that the PBP diffuses from the microcapsules into the aqueous solution, or the purified PBP composition is produced by a process comprising: mixing PBP containing organism biomass with water and a salt of divalent cations, forming a droplet, introducing a droplet of the first solution into a second solution containing a gelling agent under conditions that microcapsules form, and obtaining an extract enriched for PBP by mixing the microcapsules with a volume of an aqueous solution under conditions such that the PBP diffuses from the microcapsules into the aqueous solution or the purified PBP composition is produced by a process comprising: mixing PBP containing organism biomass with water, coating a droplet, extruded tube, sausage or some other form of this first solution with a second solution of gelling agent and introducing a droplet, extruded tube, sausage of other form of the first solution coated with the second solution into a third solution containing a salt of divalent cations under conditions such that a coated droplet, extruded tube, sausage or another form is obtained, and thereafter obtaining purified extract rich in PBP by mixing the droplets, extruded tubes, sausages or other forms with a volume of an aqueous solution under conditions such that the PBP diffuses from the droplets, extruded tubes, sausages or other forms into the aqueous solution.

In some preferred embodiments, the processes further comprise the step of drying the aqueous solution to provide a dry powder comprising PBP.

In some preferred embodiments, the PBP is characterized by one or more of the following characteristics: the a) the protein fraction of the composition comprises greater than about 30% of a protein having a molecular weight of about 17,695 kDa and an isoelectric point of about 6.29 as assayed by two dimensional gel electrophoresis followed by quantitation by densitometry; b) the protein fraction of the composition comprises greater than about 5% of a protein having a molecular weight of about 19,833 kDa and an isoelectric point of about 6.14 as assayed by two dimensional gel electrophoresis followed by quantitation by densitometry; c) the protein fraction of the composition comprises greater than about 1% of a protein having a molecular weight of about 45,578 kDa and an isoelectric point of about 6.2 as assayed by two dimensional gel electrophoresis followed by quantitation by densitometry; d) the protein fraction of the composition comprises greater than about 1% of a protein having a molecular weight of about 35,014 kDa and an isoelectric point of about 5.9 as assayed by two dimensional gel electrophoresis followed by quantitation by densitometry; e) the protein fraction of the composition comprises greater than about 0.30% of a protein having a molecular weight of about 24,688 kDa and an isoelectric point of about 5.3 as assayed by two dimensional gel electrophoresis followed by quantitation by densitometry; f) the protein fraction of the composition comprises greater than about 2% of a protein having a molecular weight of about 22,522 kDa and an isoelectric point of about 5.9 as assayed by two dimensional gel electrophoresis followed by quantitation by densitometry; g) the protein fraction of the composition comprises greater than about 1% of a protein having a molecular weight of about 21,023 kDa and an isoelectric point of about 7.3 as assayed by two dimensional gel electrophoresis followed by quantitation by densitometry; h) the protein fraction of the composition comprises greater than about 0.50% of a protein having a molecular weight of about 13,417 kDa and an isoelectric point of about 7.3 as assayed by two dimensional gel electrophoresis followed by quantitation by densitometry; i) the protein fraction of the composition comprises a ratio of major protein constituents to minor protein constituents of less than 3.5:1 based on the aggregate mass of the proteins, wherein major protein constituents are the protein having a molecular weight of about 17,695 kDa and an isoelectric point of about 6.29 as assayed by two dimensional gel electrophoresis followed by quantitation by densitometry and the protein having a molecular weight of about 19,833 kDa and an isoelectric point of about 6.14 as assayed by two dimensional gel electrophoresis followed by quantitation by densitometry and the minor protein constituents are the remainder of the proteins; j) the protein fraction of the composition comprises less than about 75% on a mass basis of the combined amounts of the protein having a molecular weight of about 17,695 kDa and an isoelectric point of about 6.29 as assayed by two dimensional gel electrophoresis followed by quantitation by densitometry and the protein having a molecular weight of about 19,833 kDa and an isoelectric point of about 6.14 as assayed by two dimensional gel electrophoresis followed by quantitation by densitometry; and k) the composition produces a solution having a color value (absorbance)of greater than 200 E 10%/1 cm when 250 mg of a dry powder of the composition are dissolved in one liter of water and absorbance is measured at 618 nm.

In some preferred embodiments, the photosynthetic microorganisms belong to the genus Arthrospira platensis. In some preferred embodiments, the microcapsules are preserved by drying, refrigeration, freezing or freeze-drying before diffusion into the aqueous medium. In some preferred embodiments, the microcapsules are packaged in a porous package with a pore size lower than microcapsules diameter, such a bag, a sachet, a dispensing pod or a similar container.

In some preferred embodiments, the present invention provides microcapsules (or a composition comprising microcapsules) immiscible in an aqueous medium, containing as dry weight, between 1 and 20 percent of jelling agent, between 1 and 20 percent of a multivalent cations salt and between 60 and 98 percent of photosynthetic microorganisms, containing at least 5 percent of PBP able to diffuse freely in the aqueous medium. In some preferred embodiments, the gelling agent is chosen among alginate and derivatives. In some preferred embodiments, the photosynthetic microorganisms belong to the genus Arthrospira platensis. In some preferred embodiments, the multivalent cations are calcium ions.

In some preferred embodiments, the present invention can be used as a food colorant conforming to Generally Accepted as Safe (GRAS) for human consumption in applications that include confectioneries, dairy products, ice creams, food color enhancements, icings and frostings and other foods and beverages and any other applications deemed safe for human or animal consumption by competent authorities.

In some preferred embodiments, the present invention provides for the use of a PBP composition, oral delivery vehicle, formulation, food or beverage, as described above to treat a disease or condition listed in Table 1. In some preferred embodiments, an effective amount of phycobiliprotein is orally administered to a subject in need thereof. In some preferred embodiments, an effective amount of phycobiliprotein is topically administered to a subject in need thereof.

In some preferred embodiments, the present invention provides methods for treating a subject in thereof, e.g., a subject having a disease or condition listed in Table 1 herein, comprising administering to the subject an effect amount of a PBP composition, oral delivery vehicle, formulation, food or beverage as described above to treat the disease or condition. In some preferred embodiments, an effective amount of PBP is orally administered to a subject in need thereof. In some preferred embodiments, an effective amount of phycobiliprotein is topically administered to a subject in need thereof.

In some preferred embodiments, the present invention provides for the use of a PBP composition for agricultural, cell culture and other applications listed in Table 1. For example, the present invention can be applied in agriculture and farming including hydroponics, aquaponics, horticulture, legumes, vegetables, row crops, grains and other forms of farming, indoor or vertical agriculture, fish farming, crustacean farming, apiculture, pig farming, chicken and other bird farming, ranching, cattle farming, farming of other domesticated and non-domesticated animals, algae culture and mushroom farming.

In some preferred embodiments, the present invention can be used in cell culture including culture of plant and animal cells and cellular agriculture (production of agricultural products using cell culture).

In some preferred embodiments, the present invention has applications in pollution control, bioremediation, environmental control and in a wide range of research and development applications in a wide range of fields.

In some preferred embodiments, the methods and techniques for the purification of PBP from Spirulina described in the Summary of Invention can be applied to purify PBP and other natural substances contained in many other algae. For example, the methods described in this invention can be used to purify Chlorella extract or Chlorella Growth Factor (CGF). CGF is a DNA and RNA-rich substance. Whereas, the final step of the PBP purification process in this invention takes place in a cool or cold aqueous solution, CGF purification requires serial diffusion steps in heated aqueous solution (up to 95 degrees centigrade).

In some preferred embodiments, the present invention provides methods of increasing a parameter of plant growth or quality comprising: applying a composition comprising PBP to a plant or plant culture in need thereof under conditions such that a parameter of plant growth or quality is improved. In some preferred embodiments, the parameter of plant growth or quality is selected from the group consisting of increased growth velocity, increased yield, increased leaf length, increased basal stem width, increased total solids (Brix), improved luminous intensity, and improved photosynthetic parameters. In some preferred embodiments, the increase in a parameter of plant growth or quality is in comparison to a control or untreated plant. In some preferred embodiments, the increase in plant growth or quality is observed in a fruit of the plant. In some preferred embodiments, the PBP is applied to the plant at a rate of from 0.1 to 100 mg/plant/day.

In some preferred embodiments, the present invention provides for use of a PBP composition as described above to increase a parameter of plant growth or quality in a plant. In some preferred embodiments, the parameter of plant growth or quality is selected from the group consisting of increased growth velocity, increased yield, increased leaf length, increased basal stem width, increased total solids (Brix), improved luminous intensity, and improved photosynthetic parameters. In some preferred embodiments, the increase in a parameter of plant growth or quality is in comparison to a control or untreated plant. In some preferred embodiments, the increase in plant growth or quality is observed in a fruit of the plant. In some preferred embodiments, the PBP is applied to the plant at a rate of from 0.1 to 100 mg/plant/day.

In some preferred embodiments, the present invention provides for the use of a PBP composition as described above as a bioremediation agent for soil and water.

In some preferred embodiments, the present invention provides for the use of a PBP composition as described above as a stimulator of plant, fungus and animal and mammalian cell grow and as proliferation agent, nutrient, medium, serum, adherence factor, scaffold, matrix or substance that can be applied in improving growth velocity, yield and efficacy in cell culture or cellular agriculture.

In some preferred embodiments, the spent microcapsules or other forms from which the PBP composition has diffused can be prepared for food and beverage applications. This includes as a complement or supplement to ingredient mixes that are designed as animal meat substitutes, analogues or copies (so called plant-based meats) that include soy, pea and other plant proteins.

In some embodiments, the present invention provides methods of bioremediation and/or soil amendment comprising: applying a composition comprising PBP to water and/or soil under conditions such that a parameter of plant growth or quality gown with or in the water or soil is improved. In some embodiments, the present invention for the use a PBP composition as described above to bioremediate and/or amend water or soil. In some embodiments, the parameter of plant growth or quality is selected from the group consisting of increased growth velocity, increased yield, increased leaf length, increased basal stem width, increased total solids (Brix), improved luminous intensity, and improved photosynthetic parameters. In some embodiments, the increase in a parameter of plant growth or quality is in comparison to a control or untreated plant. In some embodiments, the increase in plant growth or quality is observed in a fruit of the plant. In some embodiments, the PBP is applied to the soil at a rate of from 0.1 to 100 mg/plant/day. In some embodiments, the PBP is provided as microencapsulated Spirulina. In other embodiments, PBP or an algal extract according to the invention is provided in a soil amendment or bioremediation formulation at a concentration of from 0.1% to 50%, from 0.1% to 20%, or from 0.1% to 10%, where the weight percent of the PBP or algal extract in the soil amendment or bioremediation formulation is the dry weight of the algal extract added to the culture medium solution.

In some embodiments, the present invention provides methods of cell culture comprising: culturing cells in a culture medium comprising an algal extract. In some embodiments, the culture is a petri-dish culture, a roller-bottle culture or a bioreactor culture. In some embodiments, the methods further comprise the step of harvesting the cells cultured in a culture medium comprising an algal extract. In some embodiments, the algal extract is prepared by encapsulating algae to provide capsules and contacting the capsules with an aqueous medium under conditions such that a compound of interest passes from the capsuled into the aqueous solution. In some embodiments, the algal extract is a Spirulina extract or a Chlorella extract. In some embodiments, the algal extract comprises PBP. In some embodiments, the cells are mammalian cells. In some embodiments, the mammalian cells are selected from the group consisting of human, simian, porcine, bovine, ovine and poultry cells. In some embodiments, the cells are plant cells. In some embodiments, the cells are fungal cells. In some embodiments, the algal extract is provided in the medium at a concentration of from 1% to 50%, from 1% to 20%, or from 1% to 10%, where the weight percent of the algal extract in the culture medium is the dry weight of the algal extract added to the culture medium solution. In some embodiments, the algal extract is provided in the medium at a concentration of up to 50% of a 250 mg/liter solution of algal extract. In some embodiments, the algal extract is provided in the medium at a concentration of from 1 to 50% of a 250 mg/liter solution of algal extract.

In some embodiments, the present invention provides algal extracts for use in cell culture. In some embodiments, the algal extract is prepared by encapsulating algae to provide capsules and contacting the capsules with an aqueous medium under conditions such that a compound of interest passes from the capsuled into the aqueous solution. In some embodiments, the algal extract is a Spirulina extract or a Chlorella extract. In some embodiments, the algal extract comprises PBP. In some embodiments, the cells are mammalian cells. In some embodiments, the mammalian cells are selected from the group consisting of human, simian, porcine, bovine, ovine and poultry cells. In some embodiments, the cells are plant cells. In some embodiments, the cells are fungal cells.

In some embodiments, the present invention a culture medium comprising an algal extract as described above. In some embodiments, the algal extract is provided in the medium at a concentration of up to 50% of a 250 mg/liter solution of algal extract. In some embodiments, the algal extract is provided in the medium at a concentration of from 1% to 50%, from 1% to 20%, or from 1% to 10%, where the weight percent of the algal extract in the culture medium is the dry weight of the algal extract added to the culture medium solution. In some embodiments, the culture medium comprises at least a second cell culture component in addition to the algal extract. Suitable cell culture components include, but are not limited to, one or more of a carbohydrate energy source such as glucose, sucrose, dextrose or fructose, a buffer component such as HEPES, TAPS, Bicine, Tris, MOPS, TES, PIPES, sodium bicarbonate, disodium hydrogen phosphate, KH₂PO₄, sodium acetate, borate, CHES, and the like, salts such as sodium chloride, ammonium nitrate, calcium chloride, magnesium sulfate, monopotassium phosphate, potassium nitrate, boric acid, cobalt chloride, ferrous sulfate, manganese(II) sulfate, potassium iodide, sodium molybdate, zinc sulfate, copper sulfate, and salts of EDTA. In some embodiments, the algal extract is prepared by encapsulating algae to provide capsules and contacting the capsules with an aqueous medium under conditions such that a compound of interest passes from the capsuled into the aqueous solution. In some embodiments, the algal extract is a Spirulina extract or a Chlorella extract. In some embodiments, the algal extract comprises PBP. In some embodiments, the cells are mammalian cells. In some embodiments, the mammalian cells are selected from the group consisting of human, porcine, bovine, ovine and poultry cells. In some embodiments, the cells are plant cells. In some embodiments, the cells are fungal cells.

In some embodiments, the present invention provides compositions comprising microencapsulated disrupted microalgae having a protein content of from 40 to 90% dry weight and a moisture content of less than 15%. In some embodiments, the disrupted microalgae comprises microalgae with ruptured cell membranes. In some embodiments, the microalgae is selected from the group consisting of Spirulina, Chlorella, Porphyridium, AFA and Galdieria. In some embodiments, the microalgae is Spirulina. In some embodiments, the microalgae is Chlorella. In some embodiments, a phycobiliprotein or other pigment-containing protein complex has been removed from the microencapsulated disrupted microalgae. In some embodiments, the capsule of the microencapsulated disrupted microalgae comprises a gelling agent. In some embodiments, the gelling agent is an alginate. In some embodiments, the composition has a protein content of from 40% to 70% dry weight. In some embodiments, the composition has a moisture content of less than 10%.

In some embodiments, the present invention provides a food product comprising the microencapsulated disrupted microalgae described above. In some embodiments, the food product comprises at least a second food ingredient in addition to the microencapsulated disrupted microalgae. In some embodiments, the at least a second food ingredient is selected from the group consisting of a fat, carbohydrate or protein from a source different from the microencapsulated disrupted microalgae. In some embodiments, the at least a second food ingredient is selected from the group consisting of a flavoring agent or a coloring agent from a source different from the microencapsulated disrupted microalgae.

In some embodiments, the present invention provides methods of making a food product comprising combining a microencapsulated disrupted microalgae composition as described above with at least a second food ingredient. In some embodiments, the at least a second food ingredient is selected from the group consisting of a fat, carbohydrate or protein from a source different from the microencapsulated disrupted microalgae. In some embodiments, the at least a second food ingredient is selected from the group consisting of a flavoring agent or a coloring agent from a source different from the microencapsulated disrupted microalgae.

### BRIEF DESCRIPTION OF THE DRAWINGS

FIG. 1 graphically depicts the principle of obtaining microcapsules or other forms and a preferred mode of purification. The solution A (2), obtained by mixing algae biomass containing the substance to be purified, water and a jelling agent, flows through a narrow pipe or dropper (6) and forming droplets that drop into a tank filled with solution B (3) containing divalent cations. As soon as the droplets come into contact with solution B, they harden and form microcapsules that are generally round to egg-shaped.

FIG. 2 graphically depicts a preferred method of purification: microcapsules (1) are immersed in an aqueous medium (4) into which the PBP diffuses. The microcapsules may be contained in a porous package such as a sachet or bag (5).

FIG. 3 shows the kinetics of PBP extraction from microcapsules. In this example, 5 grams of fresh, wet Spirulina (corresponding to 1 gram of dry Spirulina) were purified according to the invention. The purification was done at 5° C. in 1 liter of water. PBP, in this case PC, concentration is calculated by spectrophotometry using the equations of Bennett & Bogorad. The maximum quantity of PBP is recovered after 60 h of diffusion and amounts to 10% of the dry weight of Spirulina.

FIG. 4 shows the established pH gradient in the first dimension of electrophoretic separation. This pH gradient was measured using a surface pH electrode for 3 blank IEF tube gels. The slab gel for the second dimension expands upon drying so that the final pattern is about 15.5 cm long. If SDS has been added to the sample, an SDS-NP-40 micelle migrates to the extreme acid end of the tube gel, constricting the pH gradient. In this case, the final 2-D pattern is 14.0-14.5 cm long.

FIG. 5. Two dimensional separation of a phycocyanin (PC)-rich purified extract of Spirulina (PPES) produced using the method of preparation described in this invention (Run #1) The black arrow on the 2-D gel indicates the internal standard, Tropomyosin, pI 5.2 and molecular weight 32,700. The molecular weight standard lines are due to myosin (220,000), phosphorylase A (94,000), catalase (60,000), actin (43,000), carbonic anhydrase (29,000), and lysozyme (14,000) which have been added to the sealing agarose.

FIG. 6. Two-dimensional separation of PPES (Run #2). The black arrow on the 2-D gel indicates the internal standard, Tropomyosin, pI 5.2 and molecular weight 32,700. The molecular weight standard lines are due to myosin (220,000), phosphorylase A (94,000), catalase (60,000), actin (43,000), carbonic anhydrase (29,000), and lysozyme (14,000) which have been added to the sealing agarose. Spot outlines were used for determining the spot volume.

FIG. 7. Two dimensional separation of E3 LIVE BLUE MAJIK™ (BLUE MAJIK™), a commercially available PC powder extract (Run #1) The black arrow on the 2-D gel indicates the internal standard, Tropomyosin, pI 5.2 and molecular weight 32,700. The molecular weight standard lines are due to myosin (220,000), phosphorylase A (94,000), catalase (60,000), actin (43,000), carbonic anhydrase (29,000), and lysozyme (14,000) which have been added to the sealing agarose.

FIG. 8. Two dimensional separation of BLUE MAJIK™ extract (Run #2) The black arrow on the 2-D gel indicates the internal standard, Tropomyosin, pI 5.2 and molecular weight 32,700. The molecular weight standard lines are due to myosin (220,000), phosphorylase A (94,000), catalase (60,000), actin (43,000), carbonic anhydrase (29,000), and lysozyme (14,000) which have been added to the sealing agarose. Spot outlines were used for determining the spot volume.

FIG. 9. Two dimensional separation of LINABLUE™ extract, a commercially available PC powder (Run #1) The black arrow on the 2-D gel indicates the internal standard, Tropomyosin, pI 5.2 and molecular weight 32,700. The molecular weight standard lines are due to myosin (220,000), phosphorylase A (94,000), catalase (60,000), actin (43,000), carbonic anhydrase (29,000), and lysozyme (14,000) which have been added to the sealing agarose.

FIG. 10. Two dimensional separation of LINABLUE™ extract (Run #2) The black arrow on the 2-D gel indicates the internal standard, Tropomyosin, pI 5.2 and molecular weight 32,700. The molecular weight standard lines are due to myosin (220,000), phosphorylase A (94,000), catalase (60,000), actin (43,000), carbonic anhydrase (29,000), and lysozyme (14,000) which have been added to the sealing agarose. Spot outlines were used for determining the spot volume.

FIG. 11. Percentages of proteins in the different prominent spots of PPES as compared to the equivalent spots (if present) in the other products.

FIG. 12A-E. A. Bubble Plot of MW by pI for PPES vs. BLUE MAJIK™. B. Bubble Plot of MW by pI for PPES v. LINABLUE™. C. Bubble Plot of MW by pI for LINABLUE™ v. BLUE MAJIK™. D. Bubble Plot of MW by pI for LINABLUE™ v. BLUE MAJIK™ v. PPES. E. Bubble Plot of MW by pI for PPES. The size of the bubbles is scaled to the relative percentage of the total protein represented by the particular protein spot.

FIG. 13A-C. Photomicrographs (100×) of A. PPES (A) and B. LINABLUE™ and C. Particle Analysis of PPES and LINABLUE™

FIG. 14. Lettuce cultivation prior to first transplantation

FIG. 15. PPES artificial seed containing viable plant material

FIG. 16A-B. A. Chlorella extract purified according to this invention. B. Vegan mayonnaise made using Chlorella extract purified according to this invention (mixed with paprika and Chlorella flour).

FIG. 17A-B. A. Increase in biomass following culture with PPES in Psilocybe mushrooms (A=with PPES and B=no PPES). B. Accelerated growth cycle with PPES in Malabar mushroom.

FIG. 18. Difference in biomass between PPES-bioremediated and non-PPES bioremediated corn and soy.

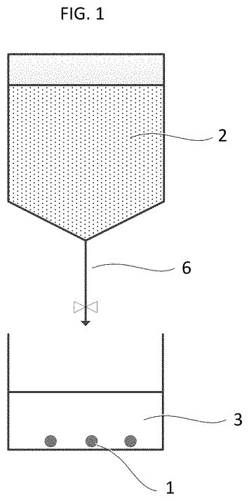

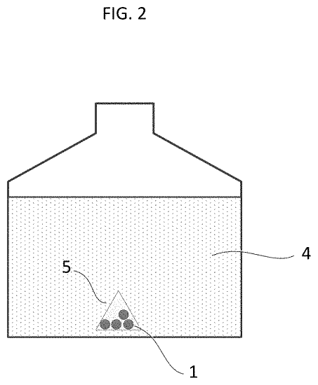

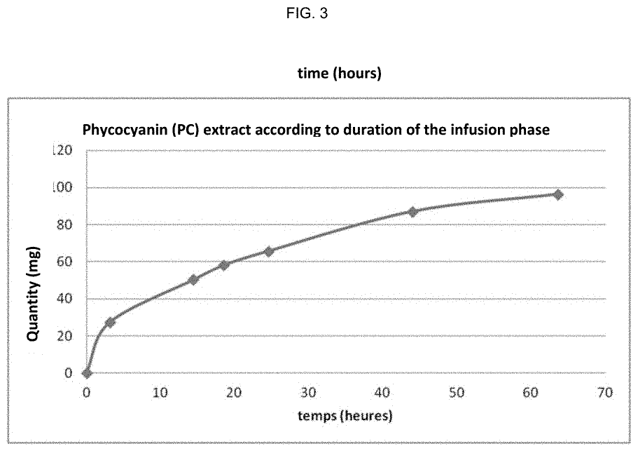

### DETAILED DESCRIPTION OF THE INVENTION

The present invention relates to the field of purification of algal extracts, methods for their preparation, the unique composition of such purified extracts and their application

The primary example of this invention relates to the field of the purification of phycocyanin (PC), a phycobiliprotein (PBP) composition, methods for its preparation, and in particular to PBP compositions with unique molecular markers that emanate from the purification process. The invention addresses the drawbacks of the prior art noted above as it provides a low-cost, high-quality and scalable purification process for the production of algal extracts, including PC in particular, an important natural food colorant for which there is growing demand. The algal extracts such as PC that are produced using this invention are characterized by unique molecular markers that are indicative of the capability of this method of purification outlined in this invention to ensure that the molecules of interest in terms of their presence, potency and activity, while concentrated through the purification process, still retain the characteristics that they had when they were present in the original material from which they were extracted and subsequently purified.

Other examples of this invention include the purification, using the methods described in this invention, of extracts of Chlorella or Chlorella Growth Factor (CGF).

The purification of PC from Spirulina is a delicate and potentially complex process, especially because these natural proteins are fragile and sensitive to heat and acids. For example, PC is known to degrade rapidly at temperatures over 55° C. and at low PH, with a loss of blue color and consequently utility as a food colorant.

The destruction of the Spirulina cell wall, an integral part of the extraction of PC, can damage the PC and if the extraction takes place over a long period cause the degradation of the PC. The nature of the raw biomass, for example whether it is fresh or dried, the type of drying process including temperature and time of exposure to heat, the preparation of the raw material, the addition of heat protecting substances, the length of time and temperature of storage, the final water content, the method of packaging, the level of oxidation of the protein and fats, exposure to bacteria and yeasts and fungi, residual chemicals and nutrients and range of other factors affect the quality and yield of PC.

Once the PC has been extracted, it has to be purified for a range of applications, especially for use as a blue natural colorant. Purification requires separation of the blue-colored PC from the green-colored chlorophylls and is usually undertaken using filtration technologies including various types of centrifuges and filtration units. This can include disk centrifuges, decanting centrifuges, charcoal filtration, ultrafiltration, nanofiltration, reverse and forward osmosis filtration, high-pressure filtration, tangential filtration and a range of technologies that separate out the PC from the chlorophyll and cell debris and other undesirable substances.

Purification using filtration is an expensive process and contributes substantially to the high current cost of PC. Purification using filtration is also wasteful as it generally requires large quantities of disposable filters or filters that require cleaning with acid and alkaline solutions that are potential environmental pollutants if not managed and disposed of appropriately. Also, in order to reduce the cost of purification using filtration, additional pre-filtration production steps may be used to precipitate out undesirable substances using chitosan and other precipitating or flocculating or purifying agents. These steps are time-consuming, expensive and potentially polluting and can damage the PC. Also, the use of certain precipitating and flocculating and purifying agents may result in the PC no longer qualifying as being of organic origin, if the PC was purified from organic biomass.

Following purification, the liquid rich in PC is generally dried using spray drying processes. Prior to spray drying sugars such as trehalose or maltodextrin and salts such as sodium citrate may be added in order to facilitate the spray drying process, protect the PC from the heat of the spray drying process and enhance the color and solubility of the powder emanating from the spray drying process. As a result of the purification and drying steps, there is an inevitable loss in the yield and quality of the PC. This impacts on the quality, color value, pricing and profitability of the final product.

Other algae extracts are also extracted and in some cased purified and in some cases dried including AFA, a source of PC. For example, Chlorella extract or Chlorella Growth Factor (CGF) is generally extracted using high temperatures from Chlorella species and generally marketed without additional purification steps. Phycoerythrin, a red-colored PBP is extracted from Porphyridium cruentum, Gracilaria or red marine algae is an extremely valuable substance used in laboratories and cosmetics that is subjected to complex and expensive purification steps. Other algae such as Hematococcus pluvialis yield oil-soluble substances such as astaxanthin through extraction steps that include homogenization.

The form of delivery of the extracted and sometimes purified algae extract depends on its applications. For example PC as a food colorant is delivered as a dry powder. For human consumption as a nutraceutical, functional food, health food or pharmaceutical, there are processes to manufacture capsules containing Spirulina or PC preparations, to be swallowed. The document CN103285375 teaches the production of PC microspheres with an external oily layer, produced by the action of a calcium solution on an emulsion containing PC and sodium alginate in the water phase, paraffin and emulsifiers. The document CN101322568 describes capsules containing Spirulina, sodium alginate, chitosan, additives and calcium chloride. PC diffusion during storage is not mentioned and does not occur because the PC is degraded by heat during the pasteurization process.

A major barrier to the more widespread use of algae extracts in human nutrition, health and agriculture is the cost of the purification process. For example, applying dried Spirulina has been shown to enhance the growth of plants and vegetables and improve yields. However, purified Spirulina extract that is rich in PC is not currently used in agriculture as it is not available in an appropriate form and quantity and price for agricultural applications. Similarly, while algae extracts have potential as natural insect repellants and for bioremediation and soil amendment, they have not been widely tested or applied due to supply and price barriers, mainly related to purification. Similarly, while algae extracts have potential to enhance animal growth (including bovine, equine, porcine and other animal farming, aquaculture, shrimp farming, aquaponics and other forms of animal farming), they are not widely used due availability and cost.

In order to avoid the high costs of purification of Chlorella to separate the chlorophyll (that has a strong green color and an unpleasant taste) from its important protein constituents, Chlorella species such as Chlorella protothecoides are currently being grown under heterotrophic conditions (where sugars replace light as a source of energy). This requires substantial investment in bioreactor technology, the use of specialized or genetically modified algae and complex culture techniques that are expensive and prone to bacterial and other contaminations. The high cost of purification is a barrier to the widespread use of algal extracts in food and beverages.

Purified algal extracts also have potential applications in cellular agriculture (the production of agricultural products from cell cultures, also described as animal or cruelty-free fish, poultry and meat farming” or laboratory-grown meats or clean meats or cell-based meats). Cellular agriculture produces both acellular products (organic molecules such as proteins and fats) and cellular products (living or once living cells such as piece of meat). For example, Chlorella and other algal extracts have been shown to have some potential in reducing the amount animal-based growth media and sera used in cell culture (see EP0049632A2)

### A. Processes of Purification

In preferred embodiments, the main processes of the present invention is based on a chemical reaction between a gelling agent and a multivalent cation. Exposing the gelling agent to the multivalent cation (or visa versa) facilitates the formation of a gel and/or a membrane, trapping the cellular debris and other large-size molecular assemblies, but allowing diffusion of water-soluble small molecules and proteins into the surrounding aqueous medium, usually water, thereby permitting the purification of the desired substance such as the PBP or PC. The multivalent cation may also act on the cell wall of, for example Spirulina, thereby making it more fragile and porous and consequently allowing the extracellular diffusion of PBP and other water-soluble molecules. Thus, the methods of the present invention result not only in the purification of, for example PBP but also permit the extraction of PBP from Spirulina in an aqueous solution in a single step. While Spriluline is specifically exemplified below, the methods described may be utilized with other specied of microalgae, including, but not limited to Chlorella, Porphyridium, Aphanizomenon flos aquae (AFA) and Galdieria. For example, the method may be used for extraction of CGF from Chlorella.

In some preferred embodiments, the processes to prepare purified PBP from Spirulina comprises the following steps:
Mixing dried or fresh Spirulina, water and a gelling agent, for example sodium alginate to provide a first solution (Solution A)
Forming microcapsules by dropping small drops of Solution A using a dropper, pipette or some other industrial device or machine into a second solution of a salt of divalent cations.
Diluting the microcapsules into a predetermined volume of water or other liquid to provide an aqueous solution to permit purification through diffusion of the PBP from the microcapsules
Removal of the microcapsules and any debris from the liquid

In some preferred embodiments, the processes to prepare purified PBP from Spirulina comprises the following steps:
Mixing dried or fresh Spirulina, water and divalent cation, for example calcium chloride to provide a first solution (Solution A)
Forming microcapsules by dropping small drops of Solution A using a dropper, pipette or some other industrial device or machine into a second solution of a gelling agent, such as sodium alginate.
Diluting the microcapsules into a predetermined volume of water or other liquid to provide an aqueous solution to permit purification through diffusion of the PBP from the microcapsules
Removal of the microcapsules and any debris from the liquid

In some preferred embodiments, the processes to prepare purified PBP from Spirulina comprises the following steps:
Mixing dried or fresh Spirulina with water
Transforming the Spirulina and water mixture into a droplet, extruded tube or sausage or some other form
Coating this droplet, extruded tube or sausage or some other form with a layer of a solution of gelling agent such as sodium alginate
Coating the droplet, extruded tube or sausage or some other form that has been coated with the gelling agent, with a salt of divalent cations.
Placing the droplets, extruded tubes or sausages or some other forms that have been coated with the gelling agent and thereafter the divalent cation solution into a predetermined volume of water or other liquid to provide an aqueous solution to permit purification through diffusion of the PBP.
Removal of the droplets, extruded tubes or sausages or some other form and any debris from the liquid.

In some preferred embodiments, the different methods previously described to produce the purified PBP compositions can be used simultaneously, sequentially or repeatedly.

In some preferred embodiments, the PBP containing organism biomass is specially prepared through heat, cold, chemical and biological processes and physical mechanisms and techniques to facilitate the purification process of this invention.

In some preferred embodiments, the PBP composition comprises a dried powder comprising a purified PBP composition as described above, the powder having a residual moisture content of less than about 10% w/w of the powder.

In some preferred embodiments, the PBP composition comprises fresh or freshly harvested PBP containing organism biomass.

In some preferred embodiments, the microcapsule or other form contains chemicals or biological agents that enhance or retard or selectively control the purification of the PBP.

In some preferred embodiments, the microcapsule or other form contains a natural substance that is purified or concentrated in conjunction with or ‘chaperoned’ into the aqueous solution by the PBP or some other substance that exists in the organism biomass.

In some preferred embodiments the aqueous solution into which the PBP diffuses is modified by changing its temperature, through agitation or mixing or the addition of chemical or biological agents such as acids, alkalis, salts and antimicrobial agents to enhance or retard diffusion of PBP and to prevent contamination.

In some preferred embodiments, the microcapsule or other form is frozen, dried or treated in some other way to enhance or retard diffusion of the PBP into the aqueous solution

In some preferred embodiments, the compositions further comprise a second biologically active agent. In some preferred embodiments, the second biologically active agent is a pharmaceutical agent. In some preferred embodiments, the second biologically active agent is a nutraceutical agent. In some preferred embodiments, the second biologically active agent is an agricultural agent. In some preferred embodiments, the second biologically active agent is a cellular culture agent for plant, meat, poultry, porcine, bovine or other animal or plant or other organism cell culture.

In some preferred embodiments, the present invention provides an oral delivery vehicle comprising a PBP composition as described above. In some preferred embodiments, the oral delivery vehicle is selected from the group consisting of a capsule, a tablet and a gummi gel.

In some preferred embodiments, the present invention provides a delivery vehicle for agricultural applications and other applications such as bioremediation and soil amendment. In some preferred embodiments the delivery vehicle is a matrix or framework including natural and artificial matrices and frameworks including mycelia, fungal carpets, combinations of bacteria and other natural substances including ‘sympathetic combinations of bacteria and yeasts’, matrices and frameworks based on nanotechnology, naturally existing plant matrices, collagen, bone and other tissue matrices and other natural or artificial materials to which the PBP composition can be attached, stuck, incorporated, bound or combined.

In some preferred embodiments, the microcapsule or other form contains, or is coated with, chemicals or biological agents that enhance or retard or selectively control the purification of the phycobiliprotein.

In some preferred embodiments, the microcapsule or other form contains a natural substance that is purified or concentrated in conjunction with or ‘chaperoned’ into the aqueous solution by the PBP or some other substance that exists in the organism biomass.

In some preferred embodiments, the microcapsule or other form is frozen, dried or treated in some other way to enhance or retard diffusion of the PBP into the aqueous solution

In some preferred embodiments, the biomass used is the genus Arthrospira, and more preferably to the species Arthrospira platensis (commonly known as Spirulina). In some preferred embodiments, the gelling agent is sodium alginate which reacts with most multivalent cations and especially well with the calcium ions. Preferred sources of calcium ions are, for example, calcium chloride and calcium gluconate. The present invention is not limited to the use of calcium ions. In other embodiments, the multivalent cations may be provided by salts of manganese, magnesium, zinc, or barium.

In some preferred embodiments, the first solution is prepared with 10% to 60% Spirulina (wet weight), 0.1% to 5% sodium alginate (dry weight) and water. Several additives can be added to the solution including flavorings, color agents, preservatives, moisteners, natural antibiotics, thickeners, sugars, anti-foaming agents, salts, acids and alkalis.

In some preferred embodiments, the second solution is an aqueous solution containing multivalent cations at a concentration between 0.005 and 0.5 mole per liter, preferably between 0.05 and 0.2 mole per liter (corresponding for example to the range 5.5-22 g/L of calcium chloride). This solution can also contain flavorings, color additives, preservatives and acidifying agents.

The present invention is not limited to any particular mechanism of action. Indeed, an understanding of the mechanism of action is not necessary to practice the invention. Nevertheless, it is contemplated that the microcapsules generated by dropping the first solution into the second solution are irregular, roughly egg-shaped with a maximum dimension of less than 6 mm and have a solid texture. Other forms can include tubular or spaghetti-like shapes, sausages, disks and irregular shapes. The setting time, during which the microcapsules or other forms are immersed in the solution B, is preferably less than 1 hour.

The purification of the preferably takes place as the last step of the process. Specifically, the microcapsules are immersed in a large volume of water and the medium containing the microcapsules is kept at a low temperature (between 0 and 6° C.) for from about 1 to 60 hours. The diffusion of the purified PBP can demonstrated when the water become progressively bluer and shows a purple fluorescence when exposed to light. These observed phenomena as characteristic of the PBP complex of Spirulina.

In preferred embodiments, the microcapsules or other forms may be manufactured in a workshop or a factory and the extraction is undertaken at a consumer's home or at an industrial site or on a farm or in a laboratory.

In some preferred embodiments, microcapsules are packaged into a porous container such as a bag, a sachet, a pod or the like, with a pore size much lower than the microcapsule size to ensure that PBP can diffuse while the microcapsules remain in the container. In some preferred embodiments, the process steps are conducted at low temperatures (e.g., between 0 and 6° C.) to prevent PBP degradation or natural or chemical preservatives are added.

The process of purification described in this invention has a number of advantages over existing algal biomass purification methods. The main advantage is that it is possible to limit or even avoid the use of membrane-based filtration in the production of purified algal extracts such as PC, thereby substantially reducing the cost of production and improving the quality of such extracts. The process described also does not require the use of salts and other substances to precipitate out the PC and the consequent expense of dialysis, osmosis, gels and exchange columns to remove such salts. This invention also can increase the yield of PBP extraction and the concentration of proteins of interest in the purified extract. This invention permits the combination of extraction and purification in a single step, if desired. As a result, it is possible to make purified extract of Spirulina with very high PC concentrations. This invention permits purification of PBP without using chemical solvents or any product from animal origin, allowing the making of an PC-rich extract compatible with vegan, halal and kosher food requirements. This invention is not damaging to delicate and sensitive proteins and other molecules including PBP thereby permitting the purification of highly active natural extracts. This invention permits low-waste, low water consumption purification with positive impacts in terms of sustainability and compatibility with circular economy principles. This invention can be applied to a wide range of algae and other natural substances. This invention is easily scalable with fairly low capital expenditure costs on machinery and technologies.

In some preferred embodiments, the processes provide microcapsules or other forms with the following characteristics: suitable for an aqueous medium, containing as dry weight, between 1 and 20 percent of gelling agent, between 1 and 20 percent of a multivalent cations salt and between 60 and 98 percent of MP, containing at least 5 percent of PBP able to diffuse freely in the aqueous medium.

In some preferred embodiments, the methods and techniques for the purification of PBP from Spirulina in described the Summary of Invention can be applied to purify PBP and other natural substances contained in many other algae including Chlorella vulgaris, AFA, Galdieria sulfuraria and Porphyridium cruentum. For example, the methods described can be used to purify Chlorella extract or Chlorella Growth Factor (CGF). CGF is a DNA and RNA-rich substance. Whereas, the final step of the PBP purification process in this invention takes place in a cool or cold aqueous solution, CGF purification requires serial diffusion steps in heated aqueous solution (up to 95 degrees centigrade).

2. Compositions of the Purified PBP Extracts

The methods described herein are useful for producing compositions containing a high quality protein fraction that is enriched for PBP. The protein content and quality of the PBP purified extracts obtained by these methods differ substantially from other described PBP purified extracts, generally purified using membrane filtration methods, as demonstrated in the Examples.

Accordingly, in some preferred embodiments, the present invention provides purified PBP protein compositions characterized by one or more of the following characteristics:

a) the protein fraction of the composition comprises greater than about 30% of a protein having a molecular weight of about 17,695 kDa and an isoelectric point of about 6.29 as assayed by two dimensional gel electrophoresis followed by quantitation by densitometry;

b) the protein fraction of the composition comprises greater than about 5% of a protein having a molecular weight of about 19,833 kDa and an isoelectric point of about 6.14 as assayed by two dimensional gel electrophoresis followed by quantitation by densitometry;

c) the protein fraction of the composition comprises greater than about 1% of a protein having a molecular weight of about 45,578 kDa and an isoelectric point of about 6.2 as assayed by two dimensional gel electrophoresis followed by quantitation by densitometry;

d) the protein fraction of the composition comprises greater than about 1% of a protein having a molecular weight of about 35,014 kDa and an isoelectric point of about 5.9 as assayed by two dimensional gel electrophoresis followed by quantitation by densitometry;

e) the protein fraction of the composition comprises greater than about 0.30% of a protein having a molecular weight of about 24,688 kDa and an isoelectric point of about 5.3 as assayed by two dimensional gel electrophoresis followed by quantitation by densitometry;

f) the protein fraction of the composition comprises greater than about 2% of a protein having a molecular weight of about 22,522 kDa and an isoelectric point of about 5.9 as assayed by two dimensional gel electrophoresis followed by quantitation by densitometry;

g) the protein fraction of the composition comprises greater than about 1% of a protein having a molecular weight of about 21,023 kDa and an isoelectric point of about 7.3 as assayed by two dimensional gel electrophoresis followed by quantitation by densitometry;

h) the protein fraction of the composition comprises greater than about 0.50% of a protein having a molecular weight of about 13,417 kDa and an isoelectric point of about 7.3 as assayed by two dimensional gel electrophoresis followed by quantitation by densitometry;

i) the protein fraction of the composition comprises a ratio of major protein constituents to minor protein constituents of less than 3.5:1 based on the aggregate mass of the proteins, wherein major protein constituents are the protein having a molecular weight of about 17,695 kDa and an isoelectric point of about 6.29 as assayed by two dimensional gel electrophoresis followed by quantitation by densitometry and the protein having a molecular weight of about 19,833 kDa and an isoelectric point of about 6.14 as assayed by two dimensional gel electrophoresis followed by quantitation by densitometry and the minor protein constituents are the remainder of the proteins;

j) the protein fraction of the composition comprises less than about 75% on a mass basis of the combined amounts of the protein having a molecular weight of about 17,695 kDa and an isoelectric point of about 6.29 as assayed by two dimensional gel electrophoresis followed by quantitation by densitometry and the protein having a molecular weight of about 19,833 kDa and an isoelectric point of about 6.14 as assayed by two dimensional gel electrophoresis followed by quantitation by densitometry; and

k) the composition produces a solution having a color value of greater than 180 E 10%/1 cm when 250 mg of a dry powder of the composition are dissolved in one liter of water and absorbance is measured at 618 nm.

In some preferred embodiments, the composition has at least two of characteristics a, b, c, d, e, f, g, h, i, j and k. In some preferred embodiments, the composition has at least three of characteristics a, b, c, d, e, f, g, h, i, j and k. In some preferred embodiments, the composition has at least four of characteristics a, b, c, d, e, f, g, h, i, j and k. In some preferred embodiments, the composition has at least five of characteristics a, b, c, d, e, f, g, h, i, j and k. In some preferred embodiments, the composition has at least six of characteristics a, b, c, d, e, f, g, h, i, j and k. In some preferred embodiments, the composition has at least seven of characteristics a, b, c, d, e, f, g, h, i, j and k. In some preferred embodiments, the composition has at least eight of characteristics a, b, c, d, e, f, g, h, i, j and k. In some preferred embodiments, the composition has at least nine of characteristics a, b, c, d, e, f, g, h, i, j and k. In some preferred embodiments, the composition has at least ten of characteristics a, b, c, d, e, f, g, h, i, j and k. In some preferred embodiments, the composition has all eleven characteristics a, b, c, d, e, f, g, h, i, j and k. It will be understood by those of skill in the art that in preferred embodiments, the compositions of the present invention may be identified by any subcombination of one or more of the characteristics identified above.

In some preferred embodiments, the purified phycobiliprotein composition is produced by a process comprising: mixing PBP containing organism biomass with water and gelling agent, forming a droplet, introducing a droplet of the first solution into a second solution containing a salt of divalent cations under conditions such that microcapsules form, and obtaining an extract enriched for PBP by mixing the microcapsules with a volume of an aqueous solution under conditions such that the phycobiliprotein diffuses from the microcapsules into the aqueous solution.

In some preferred embodiments, the purified PBP composition is produced by a process comprising: mixing PBP containing organism biomass with water and a salt of divalent cations, forming a droplet, introducing a droplet of the first solution into a second solution containing a gelling agent under conditions that microcapsules form, and obtaining an extract enriched for PBP by mixing the microcapsules with a volume of an aqueous solution under conditions such that the PBP diffuses from the microcapsules into the aqueous solution.

In some preferred embodiments, the purified PBP composition is produced by a process comprising: mixing PBP containing organism biomass with water, coating a droplet, extruded tube, sausage or some other form of this first solution with a second solution of gelling agent and introducing a droplet, extruded tube, sausage or some other form of the first solution coated with the second solution into a third solution containing a salt of divalent cations under conditions such that a coated droplet, extruded tube sausage or another form is obtained, and thereafter obtaining purified extract rich in PBP by mixing the droplet, extruded tube, sausage or other form with a volume of an aqueous solution under conditions such that the PBP diffuses from the droplet, extruded tube, sausage or other form into the aqueous solution.

In some preferred embodiments, the different methods previously described to produce the purified PBP compositions can be used simultaneously, sequentially or repeatedly.

In some preferred embodiments, the PBP compositions are provided as a dried powder. In some preferred embodiments, the residual moisture in the powder is less than 5%, more preferably less than 4%, and most preferably less than 3% or 1%. In other preferred embodiments, the powder may be produced by spray-drying, spray-freeze drying, refractance window drying, microwave drying, air drying, fluidized bed drying, vacuum drying, natural drying, microwave drying or foam drying the solutions of purified PBP.

Pharmaceutical and nutraceutical compositions of the present invention preferably comprise an effective amount of a PBP composition, such as the dried powder.

The phrases “pharmaceutical or physiologically acceptable” refers to molecular entities and compositions that do not produce an adverse, allergic or other untoward reaction when administered to an animal, such as, for example, a human, as appropriate. The preparation of a pharmaceutical composition that contains at least one compound or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically of physiologically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The PBP compositions of the present invention may be formulated with different types of carriers depending on whether it is to be administered in solid, liquid or aerosol form, and whether it need to be sterile for such routes of administration as injection. The PBP compositions of the present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

Upon formulation, PBP compositions of the present invention will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the PBP composition of the present invention suitable for administration is provided in a physiologically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the PBP composition is combined with the carrier in any convenient and practical manner, i.e., by solution, suspension, emulsification, admixture, encapsulation, absorption and the like. Such procedures are routine for those skilled in the art.

Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration: Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical or nutraceutical compositions may comprise, for example, at least about 0.1% of the PBP composition, such as a powdered PBP composition. In other embodiments, the PBP composition, such as a powdered PBP composition may comprise between about 2% to about 75% of the weight of the unit, or between about 25% to about 60%, for example, and any range derivable therein. Naturally, the amount of the PBP composition, such as a powdered PBP composition in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose of the PBP composition, such as a PBP powder composition, may also comprise from about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In preferred embodiments of the present invention, the PBP composition is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the PBP compositions may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as; for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the PBP compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

In a specific embodiment of the present invention, the composition is combined or mixed thoroughly with a semi-solid or solid carrier. In some preferred embodiments, the lipid compositions are incorporated into chewable matrices. Preferred chewable matrices jelly candies and gelatin-based gummi candy. Exemplary gummi candies include gummi bears, gummi worms, gummi frogs, gummi hamburgers, gummi cherries, gummi soda bottles, gummi sharks, gummi army men, gummi hippopotami, gummi lobsters, gummi watermelons, gummi octopuses, gummi apples, gummi peaches, and gummi oranges. The terms “gummi” and “gummy” are used interchangeably herein.

In some particularly preferred embodiments, the chewable matrix material is a sweetened material commonly referred to a gummy candy or jelly material. Gummy candy or jelly sweets are a broad general type of gelatin based, chewy candy. Gummy bears are the most popular and well known of the gummy candies. Other shapes are provided as well and gummy candies are sometimes combined with other forms of candy such as marshmallows and chocolates and as well made sour.

In preferred embodiments, the chewable matrix material comprises a gelling agent, which may be any physiologically tolerable gelling agent (preferably a saccharide (e.g. an oligosaccharide or polysaccharide), a protein or a glycoprotein) or combination capable of forming a soft, chewable, self-supporting chewable gel. Many such materials are known from the food and pharmaceutical industry and are discussed for example in Handbook of hydrocolloids, G O Phillips and P A Williams (Eds.), Woodhead Publishing, Cambridge, UK, 2000. The gelling agents are preferably materials capable of undergoing a sol-gel transformation, e.g. under the influence of a change in physiochemical parameters such as temperature, pH, presence of metal ions (e.g. group 1 or 2 metal ions), etc. Preferred gelling agents include gelatins, alginates and carrageenans. However, the use of gelatins is especially preferred as breakdown in the throat of trapped fragments is ensured and as cores having the desired properties may readily be produced using gelatins.

The gelatins used as gelling agents in the chewable matrix of the invention may be produced from the collagen of any mammal or the collagen of any aquatic species, however the use of gelatin from salt-water fish and in particular cold and warm water fishes is preferred. Gelatins having an amino acid content of 5 to 25% wt. are preferred, more especially those having an amino acid content of 10 to 25% wt. The gelatins will typically have a weight average molecular weight in the range 10 to 250 kDa, preferably 75 to 220 kDa, especially 80 to 200 kDa. Gelatins having no Bloom value or low Bloom values of 60-300, 150-300 and especially 90-200 are preferred. Where a gelatin of no Bloom value, e.g. a cold water fish gelatin, is used, this will typically be used together with another gelatin or other gelling agent. The combination of cold water and warm water fish gelatins is especially preferred. The gelatin will typically be present in the aqueous phase at a concentration of 1 to 50% wt., preferably 2 to 35% wt., particularly 5 to 25% wt. In the case of mixtures of gelatin and polysaccharides, the weight ratio of gelatin to polysaccharide in the aqueous phase will typically be 50:1 to 5:1, preferably 40:1 to 9:1, especially 20:1 to 10:1.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides, or combinations thereof. In certain embodiments, suppositories may be formed from mixtures containing, for example, the active ingredient in the range of about 0.5% to about 10%, and preferably about 1% to about 2%.

In further embodiments, the PBP compositions may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the PBP compositions may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the PBP compositions in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

In other preferred embodiments of the invention, the active compound may be formulated for administration via other routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-soluble based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Preferred bases for compositions for topical application include polyethylene glycol, lanolin, cold cream and petrolatum as well as any other suitable absorption, emulsion or water-soluble ointment base. Preferred topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain preferred embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

In some embodiments, the PBP compositions are formulated for oral administration with flavoring agents or sweeteners. Examples of useful flavoring include, but are not limited to, pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, walnut oil, cherry oil, cinnamon oil, clove oil, or peppermint oil; peanut butter, chocolate flavoring, vanilla cookie crumb, butterscotch or toffee. In one embodiment, the dietary supplement contains cocoa or chocolate. Emulsifiers may be added for stability of the final product. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product. In addition to the carbohydrates described above, the nutritional supplement can contain natural or artificial (preferably low calorie) sweeteners, e.g., saccharides, cyclamates, aspartamine, aspartame, acesulfame K, and/or sorbitol.

The PBP compositions of the present invention may also be delivered as nutraceuticals, dietary supplements, nutritional supplements, or functional foods.

The dietary supplement may comprise one or more inert ingredients, especially if it is desirable to limit the number of calories added to the diet by the dietary supplement. For example, the dietary supplement of the present invention may also contain optional ingredients including, for example, herbs, vitamins, minerals, enhancers, colorants, sweeteners, flavorants, inert ingredients, and the like. For example, the dietary supplement of the present invention may contain one or more of the following: asorbates (ascorbic acid, mineral ascorbate salts, rose hips, acerola, and the like), dehydroepiandosterone (DHEA), green tea (polyphenols), inositol, kelp, dulse, bioflavinoids, maltodextrin, nettles, niacin, niacinamide, rosemary, selenium, silica (silicon dioxide, silica gel, horsetail, shavegrass, and the like), spirulina, zinc, and the like. Such optional ingredients may be either naturally occurring or concentrated forms.

In some embodiments, the dietary supplements further comprise vitamins and minerals including, but not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D3; cyanocobalamin; sodium selenite; copper sulfate; vitamin A; vitamin C; inositol; potassium iodide. Suitable dosages for vitamins and minerals may be obtained, for example, by consulting the U.S. RDA guidelines.

In other embodiments, the present invention provides nutritional supplements (e.g., energy bars or meal replacement bars or beverages) comprising of the PBP compositions of the present invention. In preferred embodiments, the nutritional supplements comprise an effective amount of the components as described above. The nutritional supplement may serve as meal or snack replacement and generally provide nutrient calories. Preferably, the nutritional supplements provide carbohydrates, proteins, and fats in balanced amounts. The nutritional supplement can further comprise carbohydrate, simple, medium chain length, or polysaccharides, or a combination thereof. A simple sugar can be chosen for desirable organoleptic properties. Uncooked cornstarch is one example of a complex carbohydrate. If it is desired that it should maintain its high molecular weight structure, it should be included only in food formulations or portions thereof which are not cooked or heat processed since the heat will break down the complex carbohydrate into simple carbohydrates, wherein simple carbohydrates are mono- or disaccharides. The nutritional supplement contains, in one embodiment, combinations of sources of carbohydrate of three levels of chain length (simple, medium and complex; e.g., sucrose, maltodextrins, and uncooked cornstarch).

In still further embodiments, the present invention provides food products, prepared food products, or foodstuffs (i.e., functional foods) comprising the PBP compositions of the present invention. In preferred embodiments, the foods comprise an effective amount of the components as described above. For example, in some embodiments, beverages and solid or semi-solid foods comprising the fatty acids or derivatives thereof are provided. These forms can include, but are not limited to, beverages (e.g., soft drinks, milk and other dairy drinks, and diet drinks), baked goods, puddings, dairy products, confections, snack foods, or frozen confections or novelties (e.g., ice cream, milk shakes), prepared frozen meals, candy, snack products (e.g., chips), soups, spreads, sauces, salad dressings, prepared meat products, cheese, yogurt and any other fat or oil containing foods, and food ingredients (e.g., wheat flour).

In some preferred embodiments, the PBP compositions of the present invention are provided in a formulation with a stabilizing agent and/or antioxidant that does not naturally occur in Spirulina. Preferred stabilizing agents are described above. As used herein, the term “antioxidant” is recognized in the art and refers to synthetic or natural substances that prevent or delay the oxidative deterioration of a compound. In preferred embodiments, antioxidants that may be used in the PBP compositions of the present invention include tocopherols, flavonoids, catechins, superoxide dismutase, lecithin, gamma oryzanol; vitamins, such as vitamins A, C (ascorbic acid) and E and beta-carotene; natural components such as camosol, carnosic acid and rosmanol found in rosemary and hawthorn extract, proanthocyanidins such as those found in grapeseed or pine bark extract, and green tea extract.

In some preferred embodiments, the PBP compositions of the present invention are co-formulated or co-administered with one or more additional bioactive agents. In preferred embodiments, the one or more additional bioactive agents are selected from nutraceutical and pharmaceutical agents.

In preferred embodiments, the nutraceutical agents are selected from biologically active lipids (e.g., eicosapentaenoic acid (EPA), docosahexaenoic acid (DHA), conjugated linoleic acid, omega-3 fatty acids, polyunsaturated fatty acids, short chain fatty acids, medium chain fatty acids, in free fatty acid, triglyceride or phospholipid forms), resveratrol, coenzyme q10 (ubiquinone), melatonin, chondroitin sulfate, glucosamine, s-adenosylmethionine, astaxanthin, carnitine, tryptophan, alpha-lipoic acid, glutamine, inositol, green tea extract, flavonoids (e.g., epi-gallo catechin gallate (EGCG), epi-gallo catechin (EGC) and epi-catechin (EC), green tea extract, luteins), terpenes, gallates, quercertin, aequorin, and curcumin.

In preferred embodiments, pharmaceutical agents are selected from the group consisting of antineoplastic, antifungal, antiviral, anticonvulsant, antiepileptic, antidepressant, immunosuppressant, anti-inflammatory and erectile dysfunction drugs.

In some embodiments, exemplary antineoplastic drugs suitable for use in formulations of the present invention include, but are not limited to: 1) alkaloids, including microtubule inhibitors (e.g., vincristine, vinblastine, and vindesine, etc.), microtubule stabilizers (e.g., paclitaxel (TAXOL), and docetaxel, etc.), and chromatin function inhibitors, including topoisomerase inhibitors, such as epipodophyllotoxins (e.g., etoposide (VP-16), and teniposide (VM-26), etc.), and agents that target topoisomerase I (e.g., camptothecin and isirinotecan (CPT-11), etc.); 2) covalent DNA-binding agents (alkylating agents), including nitrogen mustards (e.g., mechlorethamine, chlorambucil, cyclophosphamide, ifosphamide, and busulfan (MYLERAN), etc.), nitrosoureas (e.g., carmustine, lomustine, and semustine, etc.), and other alkylating agents (e.g., dacarbazine, hydroxymethylmelamine, thiotepa, and mitomycin, etc.); 3) noncovalent DNA-binding agents (antitumor antibiotics), including nucleic acid inhibitors (e.g., dactinomycin (actinomycin D), etc.), anthracyclines (e.g., daunorubicin (daunomycin, and cerubidine), doxorubicin (adriamycin), and idarubicin (idamycin), etc.), anthracenediones (e.g., anthracycline analogues, such as mitoxantrone, etc.), bleomycins (BLENOXANE), etc., and plicamycin (mithramycin), etc.; 4) antimetabolites, including antifolates (e.g., methotrexate, FOLEX, and MEXATE, etc.), purine antimetabolites (e.g., 6-mercaptopurine (6-MP, PURINETHOL), 6-thioguanine (6-TG), azathioprine, acyclovir, ganciclovir, chlorodeoxyadenosine, 2-chlorodeoxyadenosine (CdA), and 2′-deoxycoformycin (pentostatin), etc.), pyrimidine antagonists (e.g., fluoropyrimidines (e.g., 5-fluorouracil (ADRUCIL), 5-fluorodeoxyuridine (FdUrd) (floxuridine)) etc.), and cytosine arabinosides (e.g., CYTOSAR (ara-C) and fludarabine, etc.); 5) enzymes, including L-asparaginase, and hydroxyurea, etc.; and 6) platinum compounds (e.g., cisplatin and carboplatin, etc.).

In some embodiments, exemplary antifungal drugs suitable for use in formulations of the present invention include, but are not limited to Nystatin, Amphotericin B, Griseofulvin, Miconazole, Ketoconazole, Terbinafine, Itraconazole, Fluconazole, Posaconazole, and Voriconazole.

In some embodiments, exemplary antiviral drugs suitable for use in formulations of the present invention include, but are not limited to Abacavir, Aciclovir, Acyclovir, Adefovir, Amantadine, Amprenavir, Ampligen, Arbidol, Atazanavir, Atripla, Boceprevir, Cidofovir, Combivir, Darunavir, Delavirdine, Didanosine, Docosanol, Edoxudine, Efavirenz, Emtricitabine, Enfuvirtide, Entecavir, Famciclovir, Fomivirsen, Fosamprenavir, Foscarnet, Fosfonet, Ganciclovir, Ibacitabine, Imunovir, Idoxuridine, Imiquimod, Indinavir, Inosine, Lamivudine, Lopinavir, Loviride, Maraviroc, Moroxydine, Methisazone, Nelfinavir, Nevirapine, Nexavir, Oseltamivir (Tamiflu), Peginterferon alfa-2a, Penciclovir, Peramivir, Pleconaril, Podophyllotoxin, Raltegravir, Ribavirin, Rimantadine, Ritonavir, Pyramidine, Saquinavir, Stavudine, Tea tree oil, Tenofovir, Tenofovir disoproxil, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza) and Zidovudine.

In some embodiments, exemplary anticonvulsant drugs suitable for use in dosage forms of the present invention include, but are not limited to pregabalin, gabapentin, carbamazepine, and oxcarbazepine.

In some embodiments, exemplary antiepileptic and anticonvulsant drugs suitable for use in formulations of the present invention include, but are not limited to pregabalin, gabapentin, carbamazepine, and oxcarbazepine and alprazolam, bretazenil, bromazepam, brotizolam, chlordiazepoxide, cinolazepam, clonazepam, clorazepate, clotiazepam, cloxazolam, delorazepam, diazepam, estazolam, etizolam, flunitrazepam, flurazapam, flutoprazepam, halazepam, ketazolam, loprazolam, lorazepam, lormetazepam, medazepam, midazolam, nemetazepam, nitrazepam, nordazepam, oxazepam, phenazepam, pinazepaam, prazepam, premazepam, quazepam, temazepam, tetrazepam, triazolam, clobazam, DMCM, flumazenil, eszopiclone, zaleplon, zolpidem, and zopiclone.

In some embodiments, exemplary antidepressant drugs suitable for use in formulations of the present invention include, but are not limited to tricyclic compounds such as bupropion, nortriptyline, desipramine, amitriptyline, amitriptylinoxide, butriptyline, clomipramine, demexiptiline, dibenzepin, dimetacrine, dosulepin/dothiepin, doxepin, imipramine, amineptine, iprindole, opipramol, tianeptine, trimipramine, imipraminoxide, lofepramine, melitracin, metapramine, nitroxazepine, noxiptiline, pipofezine, propizepine, protriptyine, and quinupramine; SNRIs such as duloxetine, venlafaxine, desvenlafaxine, milnacipran, levomilnacipran, sibutramine, bicifadine, and SEP-227162; and SSRIs such as citalopram, dapoxetine, escitalopram, fluoxetine, fluvoxamine, indalpin, paroxetine, sertraline, and zimelidine.

In some embodiments, exemplary immunosuppressant drugs suitable for use in formulations of the present invention include, but are not limited to Azathioprine, Mycophenolic acid, Leflunomide, Teriflunomide, Methotrexate, Tacrolimus, Ciclosporin, Pimecrolimus, Abetimus, Gusperimus, Thalidomide, Lenalidomide, Anakinra, Sirolimus, Everolimus, Ridaforolimus, Tesirolimus, Umirolimus, and Zotarolimus.

In some embodiments, exemplary erectile dysfunction drugs suitable for use in formulations of the present invention include, but are not limited to Tadalafil, Vardenafil, Sildenafil, Alprostadil, Papaverine, and Phentolamine.

In some embodiments, exemplary non-steroidal anti- inflammatory drugs (NSAIDs) suitable for use in formulations of the present invention include, but are not limited to Choline salicylate (Arthropan) Celecoxib (Celebrex); Diclofenac potassium (Cataflam); Diclofenac sodium (Voltaren, Voltaren XR); Diclofenac sodium with misoprostol (Arthrotec); Diflunisal (Dolobid); Etodolac (Lodine, Lodine XL); Fenoprofen calcium (Nalfon); Flurbiprofen (Ansaid); Ibuprofen (Advil, Motrin, Motrin IB, Nuprin); Indomethacin (Indocin, Indocin SR); Ketoprofen (Actron, Orudis, Orudis KT, Oruvail); Magnesium salicylate (Arthritab, Bayer Select, Doan's Pills, Magan, Mobidin, Mobogesic); Meclofenamate sodium (Meclomen); Mefenamic acid (Ponstel); Meloxicam (Mobic); Nabumetone (Relafen); Naproxen (Naprosyn, Naprelan); Naproxen sodium (Aleve, Anaprox); Oxaprozin (Daypro); Piroxicam (Feldene); Rofecoxib (Vioxx); Salsalate (Amigesic, Anaflex 750, Disalcid, Marthritic, Mono-Gesic, Salflex, Salsitab); Sodium salicylate (various generics); Sulindac (Clinoril); Tolmetin sodium (Tolectin); and Valdecoxib (Bextra).

In some embodiments, exemplary biological aagents suitable for use in formulations of the present invention include, but are not limited to Adalimumab; Certolizumab; Etanercept; Golimumab; Infliximab; Pegsunercept; Abatacept; Alefacept; Erythropoeitin; Infliximab; Trastuzumab; Ustekinumab; and Denileukin diftitox.

In some embodiments the PBP compositions can be presented for applications in the food and beverage industry as a powder or solution with or without salts, sugars, natural preservatives, chemicals or other substances.

In some embodiments the PBP compositions can be presented in high-sugar solutions used for panning or coating hard and soft candies, enrobing chocolates, preparation of sweets, candies and desserts and other applications in the confectionary, baking, food and dairy industries

In some embodiments the PBP compositions can be presented for applications in agriculture and animal nutrition in powder and liquid form with or without the addition of chemicals, vitamins, preservatives, sugars, medications, drugs, hormones, biological agents, surfactants, spreading and dissolution agents for uses that include all forms of irrigation, foliar spraying, hydroponics, animal feed and nutritional supplementation, fish feed and nutritional supplementation, fresh and sea water organism feed and nutritional supplementation, mushroom farming, mycelial growing, crustacean feed and nutritional supplementation, domestic animal food and treats and nutraceuticals and functional foods and medications, organic farming applications, biodynamic farming applications and regenerative agriculture applications.

In some embodiments the PBP compositions can be presented in liquid and powder form with or without: other algal compositions some purified according to this invention, chemicals, vitamins, preservatives, sugars, medications, drugs, hormones, biological agents, surfactants, spreading and dissolution agents for applications in bioremediation, pollution control, soil depollution, water treatment and water depollution.

In some embodiments the PBP compositions can be presented in liquid and powder form with or without: other algal compositions some purified according to this invention chemicals, vitamins, preservatives, sugars, medications, drugs, hormones, biological agents, surfactants, spreading and dissolution agents for applications in cell culture, cellular agriculture, micro-propagation and other techniques for growing cells of all living organisms including bacteria, fungi, plants, rodents, mammals, bird, fish, crustacean and other marine and terrestrial organisms.

3. Uses

The PBP compositions of the present invention have a number of uses.

In some preferred embodiments, the PBP compositions are used as a coloring agents, for example impart a blue color to a food or beverage. In these embodiments, the food oe beverage includes one or more ingredients that do not naturally occur in Spirulina. The PBP compositions are used to impart a blue color to food, confectionary and dairy products that are approved applications regarded as safe for human consumption by the United States Food and Drug Administration and European health authorities and other health authorities across the world.

In some preferred embodiments, the PBP compositions are used to enhance or create colors in foods. For example, to impart a vivid green color to mashed avocado (guacamole) or make light brown beers turn green in in color. The PBP compositions can be mixed with other algal compositions purified according to this invention in order to make a range or palette of natural colors for a range of applications including in food, beverages and nutraceuticals.

In some preferred embodiments, the PBP compositions are used as a colorant in non-food and beverage applications. For example, as a non-toxic colorant in liquid containing toys such as a toy plastic sword or saber that is filled with blue liquid and lit to glow.

In some preferred embodiments, the PBP compositions are used for their ability to fluoresce or glow under certain lighting conditions. These novelty applications include preparing alcoholic and non-alcoholic beverages in bars with specific lighting conditions.

In some preferred embodiments, the PBP compositions can be used as a replacement for other natural products. For example, the PBP compositions can be used to create a natural organic blue alcoholic beverage known as curacao which is currently made using artificial, chemical blue dye due to the near-extinction of the natural blue flower extract that was the original source of the blue color.

In other preferred embodiments, the PBP compositions find use in analytical methods and medical research, cosmetics, as food additives, and as an active nutraceutical, as functional food, as an ingredient of sports, performance, energy or other drinks or as pharmaceutical ingredient to treat a specific disease.

In other preferred embodiments, the PBP compositions find use in agriculture including as animal feed, animal medicine, animal growth stimulant, animal remedy, plant biostimulant, plant growth stimulant, fungal or mushroom growth stimulant, natural fertilizer, treatment for plants and crops, means for increasing plant resistance to disease and controlling plant disease, means of improving the flavor and sugar content of fruits and vegetables and as means for increasing growth velocity and yield in all forms of agriculture involving plants, fungi and animals.

In other preferred embodiments, the PBP compositions find use in domestic animal and pet care and pet nutrition.

In other preferred embodiments, the PBP compositions find use in bioremediation, environmental protection, waste management and pollution control.

In other preferred embodiments, the PBP compositions find use in cell culture and cellular agriculture.

In further preferred embodiments, other algae including Chlorella vulgari, AFA and Porphyridium cruentum purified using the invention have similar and additional uses to those described for the PBP compositions.

In other preferred embodiments, the spent microcapsules or other forms from which the PBP composition has diffused can be used as a “plant-based meat” ingredient or alternative (non-animal) protein source, especially for non-animal-meat burgers.

In other preferred embodiments, the PBP composition of the present invention and other algae compositions purified using the invention may also find use in the activities described in Table 1.

TABLE 1

| | | Algae or Algal | Reference (article, |
|---|---|---|---|
| | Activity | extract used | brand, patent, website) |
| Analytical methods, medical research and diagnostics | | | |
| | Unprocessed food marking (milk) using | Phycocyanin | CN105223180 |
| | fluorescence | | |
| | Medical imaging (fluorescence) | Phycocyanin | US2004209811 |
| | Fluorescent probes for immunodiagnostics and | Phycocyanin | Kronick MN (1983) |
| | immunoassays | | |
| | Fluorescent label for cell sorting | Phycocyanin | |
| | Marker for protein analysis (gel electrophoresis, | Phycocyanin | |
| | chromatography) | | |
| | Detection of cancer cells | Phycocyanin | |
| Cosmetics | | | |
| | Face masks | Phycocyanin | CN105106033 |
| | Anti-aging cosmetics | Phycocyanin | CN105055251 |
| | Stimulation of collagen production | Phycocyanin | JP5583259 |
| | Colorant in cosmetics | Phycocyanin | JPS6244523 |
| | Antimelanogenic effect (skin whitening and | Phycocyanin | Li-Chen Wu (2011) |
| | prevention of sunburn) | | |
| Food and food additives | | | |
| | Additive in food (in bread, pasta, yoghurt etc.) | Spirulina | |
| | Antioxidant beverage | Spirulina extract | Algama.fr |
| | Food colorant | Phycocyanin | MX2016008084 |
| | Spring roll wrapper | Phycocyanin | CN105660769 |
| | Fluorescent beverages | Phycocyanin | WO2015065887 |
| | Microcapsules with gum Arabica | Phycocyanin | CN104223052 |
| | Green food colorant | Phycocyanin | JP5770461 |
| | Emergency nutrition for malnourished children | Spirulina extract | |
| | Protein and vitamin source for vegetarians and | Spirulina extract | |
| | vegans | | |
| | High-Protein Gelled Food Products | Chlorella | US20160021923A1 |
| | | protothecoides | |
| | High Protein and High Fiber Algal Food | Various algae | US20100303990A1 |
| | Materials | | |
| | Food colorant source | Galdieria sulfuraria | Carfagna et al. (2016) |
| | | Phycocyanin extract | |
| | Thermostable blue food colorant | Cyanidioschyzon | Rahman et al. (2016) |
| | | merolae | |
| | Extrusion and encapsulation as food ingredient | Phycocyanin | Pan-utai et al. (2018) |
| Nutraceutical and Pharmaceutical | | | |
| | Spirulina in powder, flakes or tablets | Spirulina | Various |
| | Vitamin C mix, effervescent | Phycocyanin | CN106215175 |
| | Effervescent tablet | Phycocyanin | CN105815648 |
| | Cancer prevention agent | Phycocyanin | CN105727257 |
| | Hepatitis B prevention agent | Phycocyanin | CN105727257 |
| | Radiation hazard prevention agent | Phycocyanin | CN105725175 |
| | Antioxidant nutraceutical | Phycocyanin | CN105495153 |
| | Prevention and treatment of obesity | Spirulina extract | KR20150143914 |
| | Mix with ellagitannin | Phycocyanin | US2013202637 |
| | Promotion of stem cell nutrition | Phycocyanin | U.S. Pat. No. 8,334,131 |
| | Lowering free-radical levels | Phycocyanin | CN101928743 |
| | Increasing blood oxygenation capacity | phycocyanin | EP1874137 |
| | Enhancing recovery after exercise | Phycocyanin | EP1874137 |
| | Treatment of immune system problems | Phycocyanin | EP1874137 |
| | Reducing inflammation and joint pain | Phycocyanin | U.S. Pat. No. 7,473,427 |
| | Obesity prevention (lipase inhibition) | Phycocyanin | JP2004359638 |
| | Lowering blood cholesterol | Phycocyanin | JP3904107 |
| | Prevention of muscle damage during exercise | Spirulina extract | Lu HK (2006) |
| | Reduction of ischemic brain damage | Spirulina extract | Wang Y (2005) |
| | Inhibition of UVB-induced skin inflammation | Spirulina extract | Yoqianti F. (2014) |
| | Protection of the heart from oxidative damage | Spirulina extract | Earthrise.com |
| | Protection of the brain from oxidative damage | Spirulina extract | Earthrise.com |
| | Maintaining general health | Spirulina extract | Cerule.com |
| | Fighting the generalized inflammation | Spirulina extract | Cerule.com |
| | Increasing physical and mental vitality | Spirulina extract | Itlabo.fr |
| | Providing highly bioavailable essential nutrients | Spirulina extract | Itlabo.fr |
| | Stimulation of red blood cells formation | Spirulina extract | Itlabo.fr |
| | Avoids cramps during physical exercise | Spirulina extract | Itlabo.fr |
| | Alleviates intellectual tiredness | Phycocyanin | phycosan website |
| | Detoxification of the blood system | Phycocyanin | Algosource.com |
| | Detoxification of kidney-liver system | Phycocyanin | Algosource.com |
| | Permitting an increase in sports training intensity | Spirulina extract | Itlabo.fr |
| | Increases endurance over long periods of activity | Spirulina extract | Itlabo.fr |
| | Health and wellness jelly | Phycocyanin | CN105639556 |
| | Anti-diabetic action | Phycocyanin | CN106350562 |
| | Anemia treatment | Phycocyanin | CN101822367 |
| | Diabetes prevention | Phycocyanin | CN101716332 |
| | Inhibits the development of atherosclerosis | Phycocyanin | Li B. (2013) |
| | Lowers serum cholesterol, total cholesterol, | Phycocyanin | Sheu MJ (2013) |
| | triglyceride, LDL and transaminases levels | | |
| | Reduces cellular damage following brain infarct | Phycocyanin | Min SK (2015) |
| | Inhibition of the influenza virus | Phycocyanin | US2009042801 |
| | Modulation of the immune system | Spirulina extract | Mao TK (2000) |
| | Enhances the proliferation of immune cells | Phycocyanin | Li B. (2010) |
| | Antiviral activity against herpes | Spirulina extract | Hernandez CA (2002) |
| | Improves the ability to produce antibodies | Phycocyanin | Zhou CR (1998) |
| | Brain cancer palliative treatment | Phycocyanin | CN105476956 |
| | Adjuvant for thyroid cancer therapy | Phycocyanin | CN105287944 |
| | Combination with chemotherapy drugs | Phycocyanin | US2014314890 |
| | Combination with interferon to treat auto- | Phycocyanin | U.S. Pat. No. 8,110,182 |
| | immune diseases, allergy or cancer | | |
| | Radiation protection | Phycocyanin | Ivanova KG (2010) |
| | Chemotherapy-induced heart toxicity protection | Spirulina extract | Khan M. (2005) |
| | Chemotherapy-induced kidney toxicity protection | Spirulina extract | Khan M. (2006) |
| | Induction of cancer cell apoptosis | Phycocyanin | Li B. (2006) |
| | Induction of cancer cell apoptosis (colon cancer, | Phycocyanin | Saini MK (2012) |
| | in association with piroxicam) | | |
| | Induction of cancer cell apoptosis (leukemia) | Phycocyanin | Subhashini J (2004) |
| | Control of tumor progression and metastasis | Phycocyanin | Li B. (2009) |
| | Inhibition of angiogenesis to control tumor | Phycocyanin | Von Randen BHA |
| | progression | | (2005) |
| | Enhancement of the efficacy of retinoic acid | Phycocyanin | Li B. (2016) |
| | (ATRA) to slow tumor progression | | |
| | Enhancement efficacy of topotecan (TPT) to | Phycocyanin | Gantar M (2012) |
| | slow solid tumor progression | | |
| | Photodynamic therapy | Phycocyanin | U.S. Pat. No. 4,886,831 |
| | Enhancement of DNA repair activity | Spirulina extract | Qishen P (1988) |
| | Prevention and treatment of retinal diseases | Spirulina extract | US2017136075 |
| | Prevention and therapy of periodontal disease | Spirulina extract | TW201642884 |
| | Protection of lens epithelial cells | Phycocyanin | CN101812122 |
| | Protection from cataract | Phycocyanin | Kumari RP (2015) |
| | Neuroprotection (in a Parkinson's disease model) | Spirulina extract | Pabon MM (2012) |
| | Neuroprotection (against iron-induced toxicity) | Phycocyanin | Bermejo-Bescos P. et |
| | | | al. (2008) |
| | Neuronal protection in Alzheimer's disease | Phycocyanin | Rimbau V. (1999) |
| | Therapy for multiple sclerosis | Phycocyanin | Nemoto-Kawamura |
| | | | (2004) |
| | Kidney function recovery | Phycocyanin | TWI386216 |
| | Kidney protection against oxalate-mediated | Phycocyanin | Farooq SM (2004) |
| | renal cell injury | | |
| | Prevention of diabetic nephropathy | Phycocyanin | Zheng J. (2013) |
| | Inhibition of UDP-GDH enzyme | Phycocyanin | WO2017036528 |
| | Protection against DNA oxidative damage | Phycocyanin | Bhat BV (2001) |
| | Selective antioxidant activity (anti-COX2) | Phycocyanin | Reddy CM (2000) |
| | Oxygen free radical scavenger | Phycocyanin | Romay C. (1998) |
| | Treatment of osteoarthritis | Phycocyanin | Martinez SE (2015) |
| | Medical dressing | Spirulina extract | CN103341203 |
| | Wound healing | Phycocyanin | Gur CS (2013) |
| | Anti-anaphylaxis and anti-allergy | Phycocyanin | CN103059111 |
| | Prevention and treatment of male reproductive | Phycocyanin | CN103041369 |
| | toxicity caused by organophosphorus pesticide | | |
| | Protection against cyclophosphamide-induced | Spirulina extract | Chamorro-Cevallos |
| | mutations | | (2008) |
| | Reduction of exercise related oxidative stress | Galdieria sulfuraria | Carfagna et al. (2014) |
| | | phycocyanin extract | |
| | Agriculture | | |
| | Microalgae extract for agricultural use | Various algae | WO2016174646A1 |
| | Biomolecules from microalgae for animal feed | Various algae | Yaakob et al. (2014) |
| | and aquaculture | | |
| | Hot water Chlorella extracts as a soil amendment | Chlorella | Nakao et al. (1994) |
| | Biostimulant properties of cyanobacterial | Spirulina extract | Mogor et al. (2018) |
| | hydrolysate related to polyamines | | |
| | Seaweed product as plant biostimulant | Gracilaria edulis | Anand et al. (2018) |
| | Marine macroalgae for plant protection and | Various algae | Hamed et al. (2018) |
| | sustainable agriculture | | |
| | Improving plant growth and tolerance against | Various algae | Singh (2014) |
| | biotic and abiotic stress | | |
| | Enhancing the zinc status of plants | Spirulina extract | Anitha et al. (2016) |
| | Innoculant for pulses | Spirulina | Bhowmik et al (2010) |
| | Use in conjunction with Jatropha Curcas to | Spirulina | Agofhak-Nghemezi et |
| | promote tomato growth | | al. (2015) |
| | Microalgae polysaccharides as plant growth | Spirulina extract | Ellarrousi et al. (2016) |
| | biostimulant | | |
| | Aqueous seaweed extracts as plant growth | Various algae | Godlewska et al. |
| | biostimulant | | (2016) |
| | Biostimulant for organic farming | Spirulina | Aly et al. (2008) |
| | Fishmeal replacement in Parrot Fish diets | Spirulina | Sung-Sam et al (2013) |
| | Amelioration of lameness caused by | Phycocyanin | Taintor et al. (2014) |
| | degenerative joint disease in horses | | |
| | Cell culture | | |
| | Enhancing proliferation of cells in culture | Phycocyanin | JPS6244523 |
| | Promoting stem cells differentiation | Phycocyanin | effiplex.com |
| | Cell culture method | Chlorella, Spirulina, | EP0049632A2 |
| | | Scenedesmus extract | |
| | Method of human cell culture | Chlorella, Spirulina, | U.S. Pat. No. 4,468,460 |
| | | Scenedesmus extract | |
| | Optimization of serum-free medium for the | Spirulina extract | Kim et al. (1995) |
| | production of the scu-PA | | |
| | 3-D printed steak and burgers | Various algae | Vegnews.com (2018) |
| | Eliciting capsaicin and anthocyanin | Phycocyanin | Ramachandra-Rao et |
| | accumulation in plant cell cultures | | al. (1996) |
| | Other | | |
| | Enhancing glycol snow-melting activity | Phycocyanin | CN101921577 |
| | Bioremediation of soil contaminated with diesel | Spirulina and | Decesaro et al. (2016) |
| | and biodiesel | phycocyanin | |

In some particularly preferred embodiments, the PBP compositions find use in the treatment of diseases or conditions listed in Table 1. In these embodiments, an effective amount of the PBP composition, preferably formulated as described above, is administered to a subject in need thereof. Administration may be via oral, parenteral, buccal, topical, intranasal, and mucosal routes as well as other routes described above.

In some other preferred embodiments, algae extracts, other than the PBP compositions, purified using the invention may find use in the treatment of diseases or conditions listed in Table 1.

In these embodiments, an effective amount of the PBP composition and/or, algae extracts, other than the PBP compositions purified using the invention, preferably formulated as described above, is administered to a subject in need thereof. Administration may be via oral, parenteral, buccal, topical, intranasal, and mucosal routes as well as other routes described above.

In some other preferred embodiments, PBP compositions and other algal purified using the invention may find use in the analytical, food and food additive, nutraceutical and pharmaceutical, agriculture, cell culture and other indications, listed in Table 1.

### EXAMPLES

### Example 1

### Comparative Analysis of PBP Obtained by Methods of the Present Invention

Purpose. Spirulina is the source of natural blue extract that can be used as a colorant, food and beverage ingredient, nutraceutical, pharmaceutical, agricultural growth stimulant, agricultural nutritional product, scientific reagent, cell culture agent and a variety of other applications. A number of different purified extracts of Spirulina are available that are produced by a variety of different processes. Investigations have shown that the production processes used appeared to substantially damage, denature, remove or transform a number of the protein components in the Spirulina, the PBP complex in particular. The present invention describes a novel process aimed at preserving the important components of Spirulina intact during purification (and optionally extraction) and concentrating some of these components. The experiments described below were carried out to compare the components of the phycocyanin-rich, purified extract of Spirulina, the present invention, (referred to as PPES) with two commercially available Spirulina PBP-containing products (BLUE MAJIK™ and LINABLUE™. The experiments show that the purification process described in the present invention is substantially better at preserving, concentrating and protecting the PBP and other protein components of Spirulina than the processes used in the 2 commercially available Spirulina extract products.

Materials & Methods. Two-dimensional electrophoresis was performed according to the carrier ampholine method of isoelectric focusing (O'Farrell, P. H., J. Biol. Chem. 250: 4007-4021, 1975, Burgess-Cassler, A., Johansen, J., Santek, D., Ide J., and Kendrick N., Clin. Chem. 35: 2297, 1989. Isoelectric focusing was carried out in a glass tube of inner diameter 2.3 mm using 2.0% pH 3-10 isodalt Servalytes (Serva, Heidelberg, Germany) for 9,600 volt-hrs. One μg of an IEF internal standard, tropomyosin, was added to each sample. This protein migrates as a doublet with lower polypeptide spot of MW 33,000 and pH 5.2. The enclosed tube gel pH gradient plot for this set of Servalytes was determined with a surface pH electrode. After equilibration for 10 min in buffer ‘O’ (10% glycerol, 50 mM dithiothreitol, 2.3% SDS and 0.0625 M tris, pH 6.8), each tube gel was sealed to the top of a stacking gel that overlaid a 10% acrylamide slab gel (0.75 mm thick). SDS slab gel electrophoresis was carried out for about 4 hrs at 15 mA/gel. The following proteins (MilliporeSigma) were used as molecular weight standards: myosin (220,000), phosphorylase A (94,000), catalase (60,000), actin (43,000), carbonic anhydrase (29,000), and lysozyme (14,000). These standards appear as bands at the basic edge of the Coomassie Brilliant Blue R-250-stained 10% acrylamide slab gel. The Coomassie Blue-stained gels were dried between sheets of cellophane with the acid edge to the left.

Computerized Comparisons. The Coomassie blue-stained gels obtained from the samples were scanned with a linear densitometer GE ImageScanner 3). The scanner was checked for linearity prior to scanning with a calibrated Neutral Density Filter Set (Melles Griot, Irvine, Calif.). The images were analyzed using Progenesis Same Spots software (version 4.5, 2011, Total Lab, UK) and Progenesis PG240 software (version 2006, Total Lab, UK). The general method of computerized analysis for these pairs included image warping in conjunction with detailed manual checking. Spot % is equal to spot integrated density above background (volume) expressed as a percentage of total density above background of all spots measured.

MW and pI Measurements. Note that the isoelectric point (pI) measurements are approximate being based on the pH gradient plot found on the next page for this batch of ampholines for conditions of 9M urea and room temperature of 22° C. Since the samples themselves may perturb the pH gradient, internal pI standards should be included if more exact pI measurements are required. The molecular weight and pI values for each spot are determined from algorithms applied to the reference image.

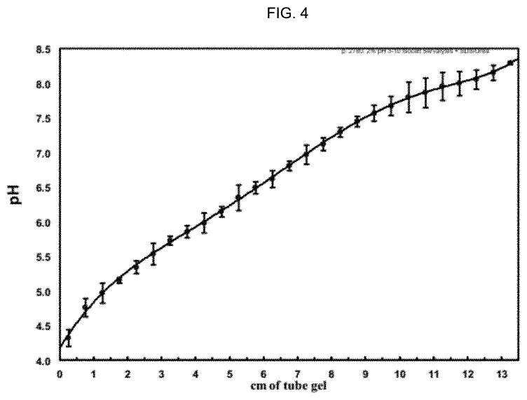

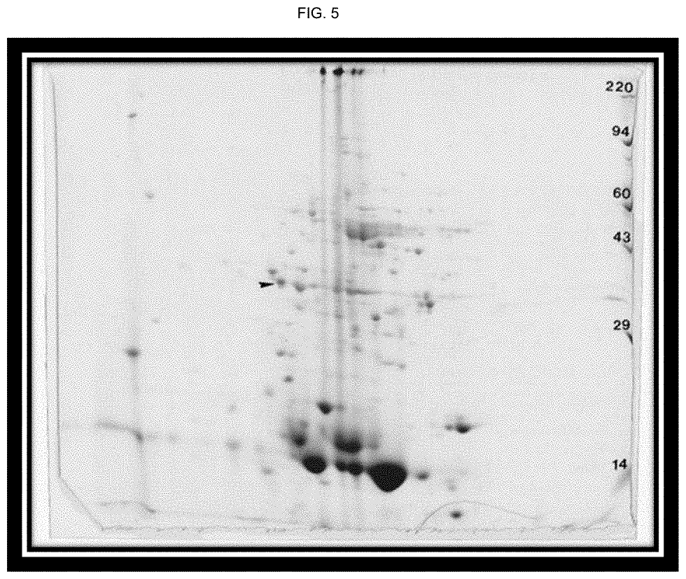

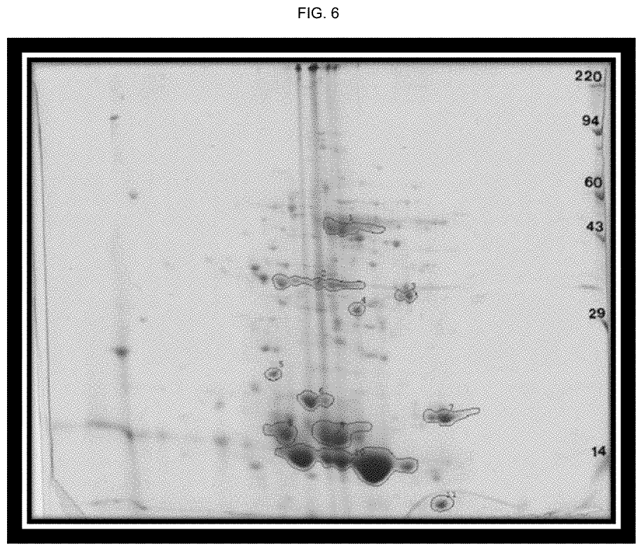

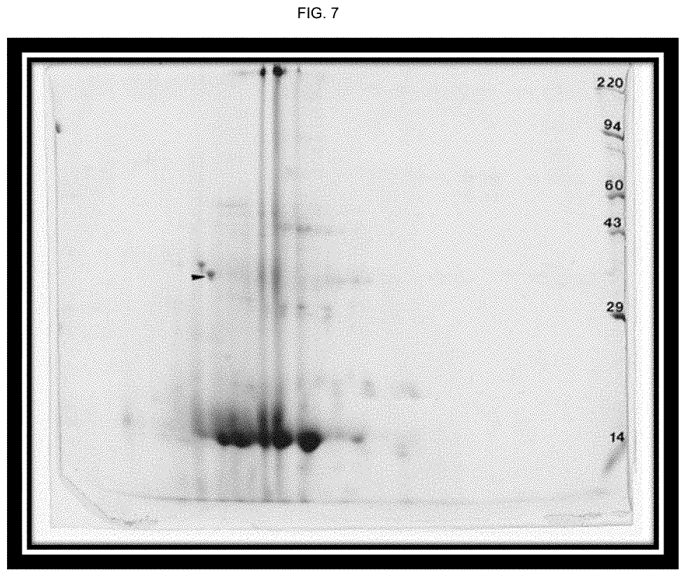

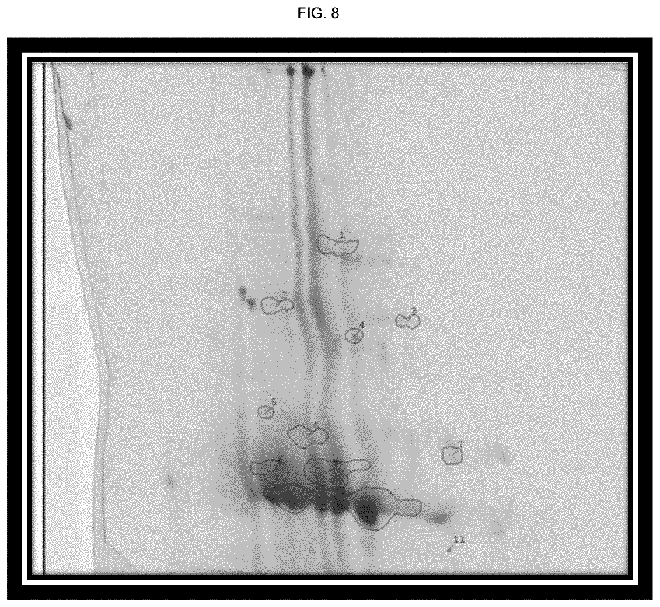

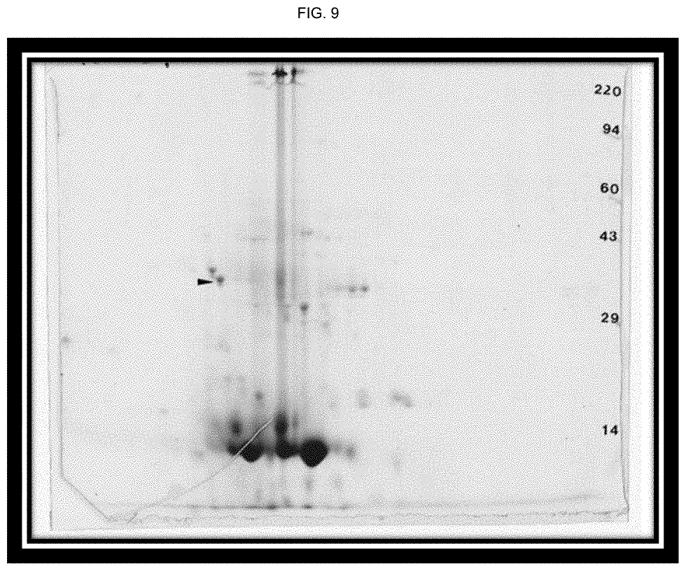

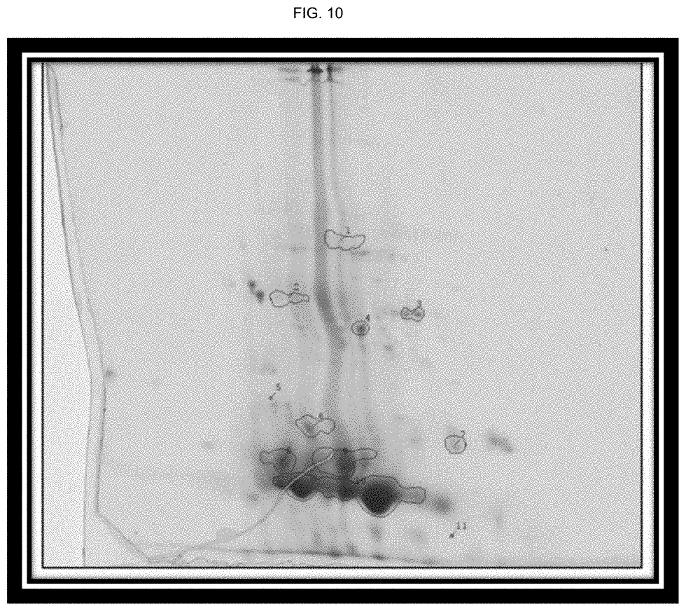

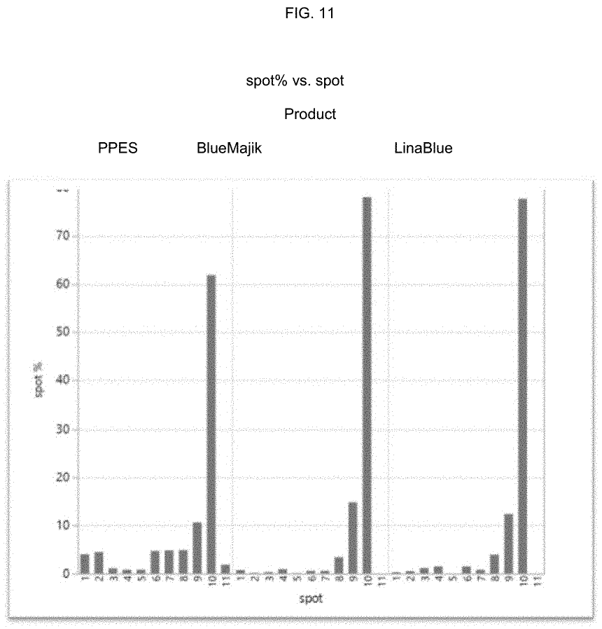

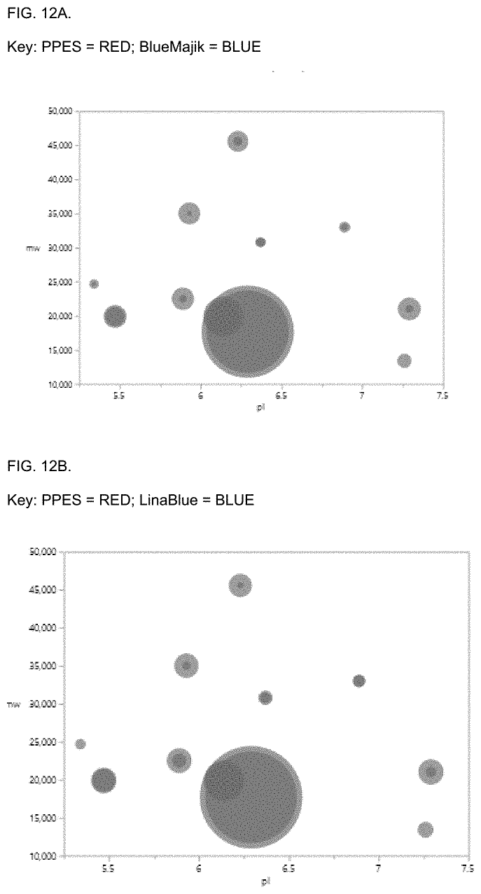

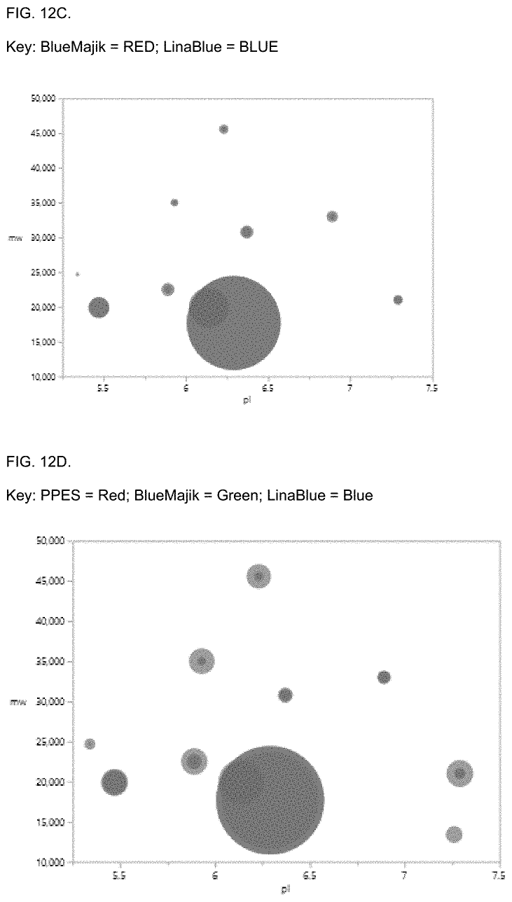

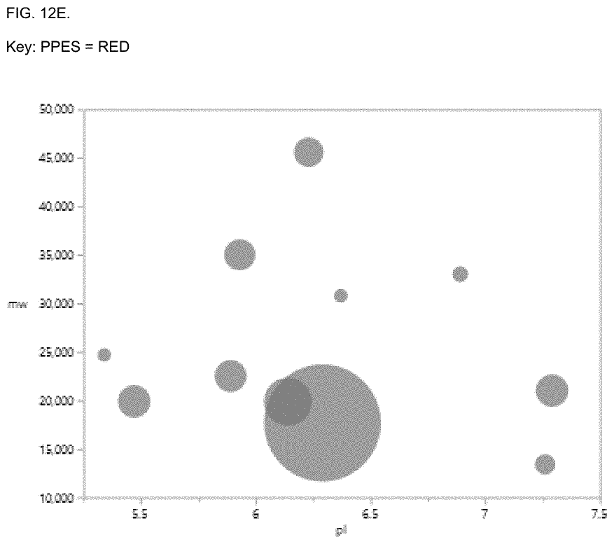

The results of the analysis are provided in FIGS. 4-12 and Table 2.

TABLE 2
Isoelectric point, estimated molecular weight, spot volume and
spot percentages of the eleven most prominent proteins
in PPES, BLUE MAJIK ™ (BM), and LINABLUE ™ (LB).

| spot | pI | | | mw | | | volume | | | spot % | | |
|---|---|---|---|---|---|---|---|---|---|---|---|---|
| Product | PPES | BM | LB | PPES | BM | LB | PPES | BM | LB | PPES | BM | LB |
| 1 | 6.23 | 6.2 | 6.2 | 45578 | 45578 | 45578 | 197.7 | 19.6 | 10.7 | 4.02 | 0.81 | 0.32 |
| 2 | 5.93 | 5.9 | 5.9 | 35014 | 35014 | 35014 | 217.3 | 5.6 | 17.7 | 4.42 | 0.23 | 0.53 |
| 3 | 6.89 | 6.9 | 6.9 | 33008 | 33008 | 33008 | 56 | 9.6 | 40.3 | 1.14 | 0.39 | 1.2 |
| 4 | 6.37 | 6.4 | 6.4 | 30793 | 30793 | 30793 | 42.5 | 23 | 50.8 | 0.86 | 0.95 | 1.52 |
| 5 | 5.34 | 5.3 | 5.3 | 24688 | 24688 | 24688 | 42.2 | 3.5 | | 0.86 | 0.14 | |
| 6 | 5.89 | 5.9 | 5.9 | 22522 | 22522 | 22522 | 229.1 | 14 | 51.5 | 4.66 | 0.58 | 1.54 |
| 7 | 7.29 | 7.3 | 7.3 | 21023 | 21023 | 21023 | 236.3 | 15.3 | 28.7 | 4.81 | 0.63 | 0.86 |
| 8 | 5.47 | 5.5 | 5.5 | 19912 | 19912 | 19912 | 238.3 | 83.4 | 133.6 | 4.85 | 3.44 | 3.99 |
| 9 | 6.14 | 6.1 | 6.1 | 19883 | 19883 | 19883 | 521 | 357.5 | 412.5 | 10.59 | 14.75 | 12.32 |
| 10 | 6.29 | 6.3 | 6.3 | 17695 | 17695 | 17695 | 3045.7 | 1892.1 | 2602 | 61.93 | 78.07 | 77.72 |
| 11 | 7.26 | 7.3 | 7.3 | 13417 | 13417 | 13417 | 91.8 | | | 1.87 | | |

Note
that in some cases prominent spots in PPES are not present in the other products.

Based on visual inspection of the electrophoretic patterns, the purified extracts compared are quite different. The relative quantities of the component proteins are obviously very different. The two comparative products (BLUE MAJIK™ and LINABLUE™) each had a substantial amount of insoluble material that was presumably denatured proteins, substances related to their production process and other cellular components that had to be removed by centrifugation to carry out the electrophoretic analysis. Two electrophoresis runs were made and more vigorous attempts were made to solubilize the samples to obtain cleaner, low background gels. Despite these efforts, there were fractions of the proteins that remained in the stacking gel and did not enter the separating gel. This suggests that a portion of the proteins have been denatured in the production process. The protein remaining in the stacking gel results in a distortion of the electric field that has an effect giving rise to the streaking pattern. What is very apparent by inspection of the gel patterns is that PPES has a number of proteins that are not present at all in the other two products. Overall, the goal was to assess the relative quantity of the different proteins in the compared products. Because some of the proteins in PPES were not present in the compared samples we chose 11 proteins from PPES as the primary reference. The areas used to determine the colorimetric volume are indicated by the outlines.

Specific differences between PPES and the comparative products include the following, where calculations of are based on spot area multiplied by color intensity as proportional to mass:
The first major protein in PPES constitutes approx. 62% of the total protein (Spot #10) and has molecular weight of 17,695 and an isolectric point of 6.29 and is present in the comparative products.
The second ranked major protein in PPES (Spot #9) constitutes about 10.6% of the total has molecular weight of 19883 and an isolectric point of 6.14 and is present in the comparative products.
PPES contains multiple additional proteins present at higher concentrations than in either comparator product (the secondary proteins):
Spot 1: Constitutes 4.02% of PPES, having a molecular weight of 45,758 and an isoelectric point of 6.23; this spot is less than 0.8% of the comparators
Spot 2: Constitutes 4.42% of PPES, having a molecular weight of 35014 and an isoelectric point of 5.93; this spot is less than 0.5% of the comparators
Spot 6: Constitutes 4.66% of PPES, having a molecular weight of 22522 and an isoelectric point of 5.89; this spot is less than 1.54% of the comparators
Spot 7: Constitutes 4.81% of PPES, having a molecular weight of 21023 and an isoelectric point of 7.29; this spot is less than 0.9% of the comparators
Spot 8: Constitutes 4.85% of PPES, having a molecular weight of 19912 and an isoelectric point of 5.47; this spot is less than 4% of the comparators
The ratio of the aggregate masses of the 5 secondary proteins to the 2 major proteins (Spot 10+Spot 9) in PPES is 0.31, but only 0.06 and 0.08 in the comparator proteins.
PPES contains two proteins absent from one or more comparator products (the missing proteins):
Spot 5: Constitutes 0.86% of PPES, having a molecular weight of 24688 and an isoelectric point of 5.34; this spot is less than 0.15% of one comparator and absent from the other
Spot 11: Constitutes 1.87% of PPES, having a molecular weight of 13417 and an isoelectric point of 7.26; this spot is absent in both the comparators
All of the other proteins enumerated in PPES are present in a considerably higher content than in the comparators.
The comparator products have much more degraded low molecular weight proteins running at the electrophoretic front.
The comparator products have a relatively large of precipitate that has to be removed by centrifugation.
The ratio of the aggregate masses of the “Minor proteins” comprising the secondary proteins plus the missing proteins, as defined above, comprise 0.37 of PPES but less than 0.10% of the comparators

### Example 2

### Physical Differences between PPES and LINABLUE™

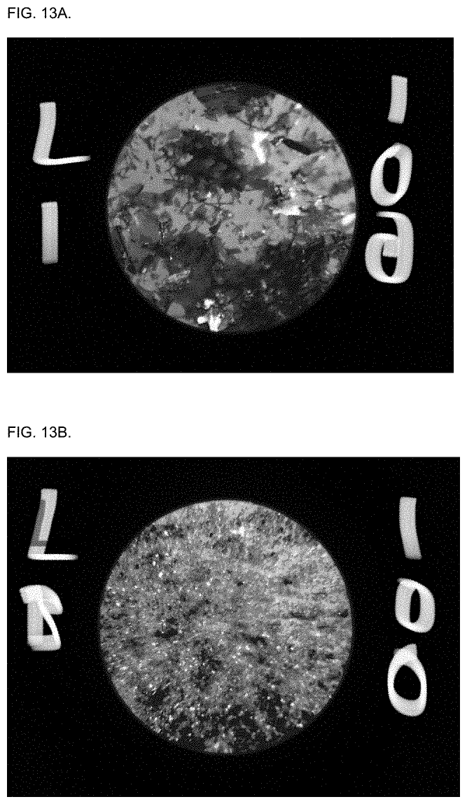

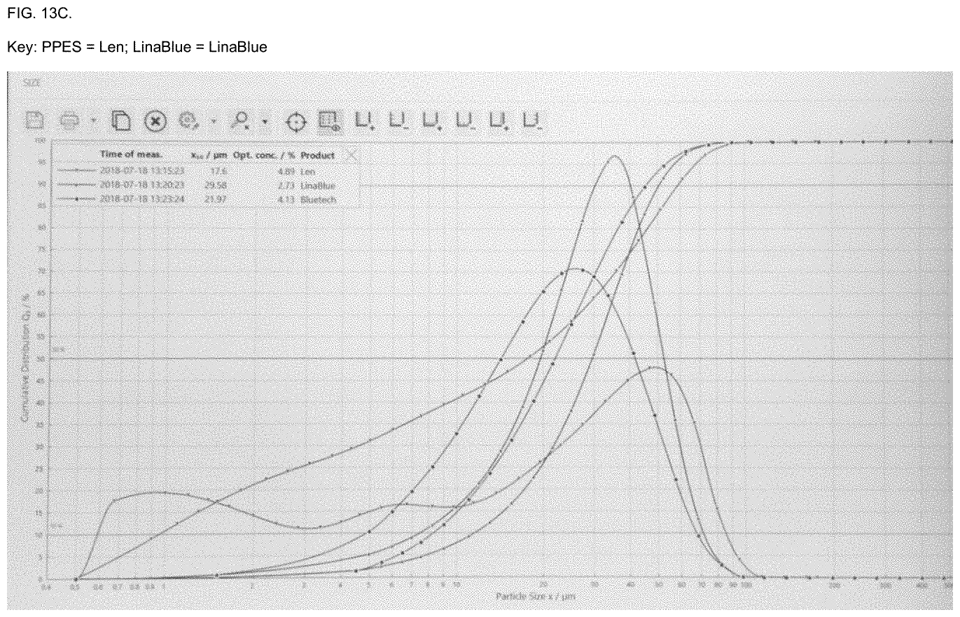

Samples of PPES, a PC-rich purified Spirulina extract preparation according to the present invention, and LINABLUE™ were compared by color photomicrography at a magnification of 100 times (100×). The results are presented in FIGS. 13A (PPES) and 13B (LINABLUE™). As can be seen, the PPES is characterized as containing pure PBP flakes while the LINABLUE™ has a granular appearance, probably due to residues of compounds other than PBP.

Samples of PPES, a PBP preparation according to the present invention and LINABLUE™ were compared using dry dispersion particle analysis (Sympatec Partikel Technik GmbH). The results are presented in FIG. 13C. As can be seen, PPES shows a uniform, homogenous particle structure pattern whereas LINABLUE™ shows a particle structure pattern indicative of possible protein degradation and the presence of sugars and salts.

### Example 3

### Application of PC-Rich, Purified Extract of Spirulina Purified According to this Invention (PPES) as an Agent to Protect Against Sunburn

In the first example, a number of individuals all consumed approximately not less that 250 mg of PPES in a lyophilized powder form per day. These individuals were fair skinned and prone to sunburn. All reported an almost immediate improvement in their resistance to sunburn within 6 hours of consuming the first dose of PPES.

In the second example, 4 volunteer subjects were exposed to a minimal erythematous dose (MED) and MED doubled (600/1200 or 800/1600) using a UV-B source namely a NewSurg Targeted Narrow Band UV-B device on the volar forearm. The individual phototypes were I and II and each skin site acted as its own control in measurement of basal skin color. Readings of erythema was taken for all exposure sites using chromameter and digital photographs were taken. The MED was defined as the lowest dose that produces pink erythema with distinct borders. Quantitative measurement of skin color was undertaken using a NIX chromameter. This device measures the erythema and skin color based on Commission International de I'eclairage L*a*b* color space. The L*a*b* color space method (CIELAB), developed in 1976, is the most frequently used to objectively assess colors. In this system the L* coordinate correlates with the intensity of the reflected light (brightness) and the a* and b* coordinates are chromatic, covering the spectrum from red to green and from yellow to blue, respectively. The a* value is well recognized to linearly correlate with skin erythema. On Day 1, determination of MED and MED*2 was undertaken where after the 4 individuals consumed either 250 mg of PPES in cold milk coffee or only cold milk coffee. This regimen was repeated 3 times—late afternoon, evening and next morning. On the following day, repeat MED and 2MED was administered. Clinical photography and chromameter readings were undertaken at 1-hour post-exposure and longer intervals. CIELAB results: Mean 60 minutes following exposure (MED*2)—13 (pre-PPES) and 9 (post-PPES); Mean 12 hours following exposure (MED*2)—19 (pre-PPES) and 17 (post-PPES); Mean/SD for 5 day follow-up (1 subject, MED*2) based on twice daily readings—22.85/3.34 (pre-PPES) and 19.42/3.41 (post-PPES). P=0.08 and difference of 15%. General observations included a marked difference in skin sensitivity, itching and pain and less skin peeling and more rapid skin recovery in the PPES group. This small experiment provides some evidence of the potential of PPES as an oral sun protection and sunburn recovery nutraceutical. Of particular interest is the protective and pain reduction potential at high UV doses, possibly indicating some of the dermal protective functions mentioned in the basic science literature, which may justify testing in radiation oncology and perhaps pain management for severe burns. Another observation of interest is the rapidity of action and the effectiveness of acute dosing.

### Example 4

Application of this PC-Rich, Purified Extract of Spirulina Purified According to this Invention (PPES) as a Stimulant to Enhance the Growth, Yield and Flavor and Shelf Life of Plants

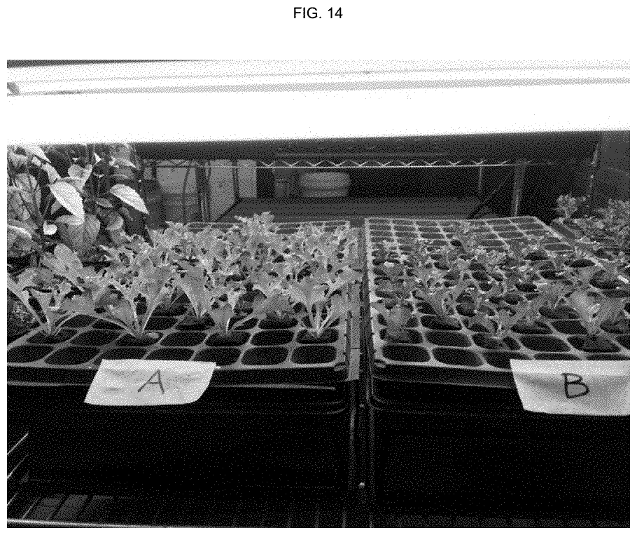

A series of indoor and outdoor tests were conducted whereby plants (basil, tomatoes, peppers, lettuce and medical cannabis (grown at a legal facility) were divided into experimental and control groups. The control (NPK solution) and experimental groups (NPK plus PPES solution) were grown under similar conditions, in accordance with current best practice for indoor and outdoor agriculture with the only difference between the 2 groups being that the experimental group was given at least 1 mg/plant/day of PPES through hydroponic, irrigation or foliar spraying methods. The experimental and control groups were compared phenotypically and based on yield, sugar content and photosynthetic performance. Specific parameters measured included growth velocity, leaf length, basal stem width, yield, leaf photometry, fluorometry, and Brix (total solids). Some examples of results include: a minimum of 30% increase in growth velocity in all plants given the PPES as compared to the control groups; a minimum of 10% improvement in Maximal Quantum Yield (of photosynthesis representing the ability of photosystem II to transform light into electrons in the photosynthetic chain) and the Performance Index (an aggregate parameter of photosynthesis representing the overall balance of absorption of light, quantum yield of photosynthesis and electron transfer in the photosynthetic chain) in all plants given the PPES extract as compared to the control groups; a minimum of 10% increase in total solids (Brix) in all plants given PPES as compared to the control groups; a minimum of 20% increase in overall root mass in all plants given the PPES as compared to the control groups; a minimum of 25% increase in stem diameter in all plants given the PPES as compared to the control groups; a minimum of 15% increase in leaf length in all plants given the PPES as compared to the control groups; a minimum of 20% increase in height in all plants given PPES as compared to the control groups; a minimum of 15% increase in yield in all plants given the PPES as compared to the control groups; a minimum gain of 4 days time to readiness for harvest in all plants given the PPES as compared to the control groups and observable improvements in the taste, quality, shelf life and color of the harvested production in all plants given the PPES as compared to the control groups. An observable difference in the intensity of the green leaf color was seen in all plants given the PPES as compared to the control groups. FIG. 14. shows the difference between lettuce plants (prior to first transplantation) given the PPES as compared to the control group.

The following Tables contain data collected on red lettuce and demonstrate a 55% increase in leaf length and basal stem width in treated plants (NPK plus PPES, designated BYAS-A601 in the tables) as compared to control plants (NPK only).

| | | End of | | | End of |
|---|---|---|---|---|---|
| BYAS-A601 | Transplant | Trial | CONTROL | Transplant | Trial |
| Avg. Leaf | 5.43 | 12.80 | Avg. Leaf | 2.69 | 6.24 |
| length cm | | | length cm | | |
| Avg. Basal | 0.22 | 6.53 | Avg. Basal | 0.17 | 2.25 |
| stem width cm | | | stem width cm | | |

The following Tables contain data collected on red lettuce and demonstrate a 15% increase in yield and 28% difference in growth velocity in treated plants (NPK plus PPES) as compared to control plants (NPK only).

| BYAS-A601 | | CONTROL | |
|---|---|---|---|
| 20 days to harvest | 1.6 | 28 days to harvest | 1.37 |
| Yield-kg | | Yield-kg | |
| Avg. Yield per Plant | 0.06 | Avg. Yield per Plant | 0.05 |

The following Tables contain data collected on red lettuce and demonstrate a 23% difference in photosynthetic efficiency in treated plants (NPK plus PPES) as compared to control plants (NPK only).

| BYAS-A601 | | CONTROL | |
|---|---|---|---|
| Maximal quantum yield | 0.801 | Maximal quantum yield | 0.797 |
| of photosyntesis | | of photosyntesis | |
| Performance index | 2.78 | Performance index | 2.197 |

The following Tables contain data collected on tomatoes and demonstrate a 20% increase in leaf length and basal stem width in treated plants (NPK plus PPES) as compared to control plants (NPK only).

| | | End of | | | End of |
|---|---|---|---|---|---|
| BYAS-A601 | Transplant | Trial | CONTROL | Transplant | Trial |
| Avg. Leaf | 9.42 | 14.39 | Avg. Leaf | 9.42 | 10.86 |
| length cm | | | length cm | | |
| Avg. Basal | 0.76 | 3.72 | Avg. Basal | 0.76 | 2.93 |
| stem width cm | | | stem width cm | | |

The following Tables contain data collected on tomatoes and demonstrate a 40% increase in yield in treated plants (NPK plus PPES) as compared to control plants (NPK only).

| BYAS-A601 | | CONTROL | |
|---|---|---|---|
| 20 weeks Yield-kg | 15.88 | 20 weeks Yield-kg | 10.21 |
| Avg. Yield per Plant | 1.31 | Avg. Yield per Plant | 0.84 |

The following Tables contain data collected on tomatoes and demonstrate a 15% difference in photosynthetic efficiency in treated plants (NPK plus PPES) as compared to control plants (NPK only).

| BYAS-A601 | | CONTROL | |
|---|---|---|---|
| Maximal quantum yield | 0.678 | Maximal quantum yield | 0.564 |
| of photosyntesis | | of photosyntesis | |
| Performance index | 1.039 | Performance index | 0.892 |

The following Tables contain data collected on green peppers and demonstrate a 30% increase in leaf length and basal stem width in treated plants (NPK plus PPES) as compared to control plants (NPK only).

| | | End of | | | End of |
|---|---|---|---|---|---|
| BYAS-A601 | Transplant | Trial | CONTROL | Transplant | Trial |
| Avg. Leaf | 6.59 | 12.39 | Avg. Leaf | 6.59 | 9.43 |
| length cm | | | length cm | | |
| Avg. Basal | 0.51 | 3.48 | Avg. Basal | 0.51 | 2.56 |
| stem width cm | | | stem width cm | | |

The following Tables contain data collected on tomatoes and demonstrate a 40% increase in yield in treated plants (NPK plus PPES) as compared to control plants (NPK only).

| BYAS-A601 | | CONTROL | |
|---|---|---|---|
| 20 weeks Yield-kg | 14.54 | 20 weeks Yield-kg | 7.98 |
| Avg. Yield per Plant | 1.21 | Avg. Yield per Plant | 0.66 |

The following Tables contain data collected on green peppers and demonstrate a 23% difference in photosynthetic efficiency in treated plants (NPK plus PPES) as compared to control plants (NPK only).

| BYAS-A601 | | CONTROL | |
|---|---|---|---|
| Maximal quantum yield | 0.755 | Maximal quantum yield | 0.681 |
| of photosyntesis | | of photosyntesis | |
| Performance index | 1.071 | Performance index | 0.850 |

Quality was assessed by Brix measurements and determination of luminous intensity by photometry. Brix testing demonstrated that treated lettuce showed a 50% increase in total solids as compared to control, treated green bell pepper demonstrated a 50% increase in total solids as compared to controls, and treated tomatoes showed a 40% increase in total solids as compared to controls. Improvements were also seen in luminous intensity as compared to controls.

A further series of tests were conducted on “row crops”, namely soy and corn grown outdoors. It was noted that the corn and soy plants that received a single dose of PPES when they were planted showed good early growth response (soy) and an increase in stem diameter, plant fresh weight, plant height and yield (corn), as compared to a control group that did not receive PPES.

### Example 5

Application of this PC-rich, Purified Extract of Spirulina Purified According to this Invention (PPES) to Increase Vitality and Resistance to Cold Weather, and to Improve Sporting Performance, Sporting Endurance and Recovery from Exercise

Here the individuals all consumed not less that 250 mg of PPES purified according to in a lyophilized powder form per day.

Case 1: A woman in late middle-age who owned and managed a cheese shop in a suburb of Paris, France. The shop was open to the elements and extremely cold in the winter. The woman complained that the cold was almost incapacitating even though she dressed as warmly as she could. The woman was of slight build, of normal Body Mass Index (BMI) and was extremely fit, being a regular long-distance runner. Almost immediately after commencing the daily ingestion of the PPES, the woman noticed that her resistance to cold and general vitality in cold weather improved substantially. The woman took the PPES over an entire winter without noticing any diminution in its positive effects.

Case 2: A middle-aged open water swimmer began consuming PPES during alternate weeks while training for an event. The swimmer of normal build and good health observed that during the week periods that he consumed PPES his performance and endurance was improved as compared to the weeks when he did not consume the PPES. The swimmer also found that mixing small amounts of the PPES with his sports hydration drink assisted his recovery following vigorous exercise. Neither Case 1 nor Case 2 observed any adverse effects from taking the PPES. Neither Case 1 nor Case 2 observed any weight gain while taking the PPES.

### Example 6

### Application of PC-Rich, Purified Extract of Spirulina Purified According to this Invention (PPES) to Accelerate and Improve Cell Culture and Propagation of Plant Material

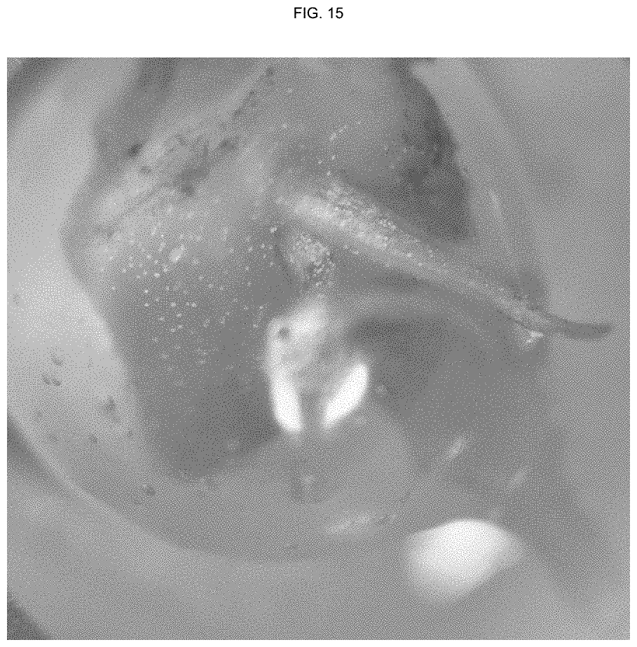

Here the in-vitro propagation of homogenized axillary bud cells of Cannabis Sativa undertaken using cell culture and synthetic seed technology, was undertaken. Axillary bud cells of Cannabis Sativa were successfully encapsulated and cultured in calcium alginate beads. The cells were cultured in alginate beads containing Murashige and Skoog medium supplemented with thidiazuron and Plant Preservative Mixture (PPM). The addition of PPES (this invention) into the beads had a positive effect on the speed of cell culture and overall plantlet development. Following rootlet development, transfer took place onto coco natural growth medium with the PPES containing synthetic seeds showing improved survival as compared those that did not contain the PPES. Similar results were found when PPES was added to the medium in which the synthetic seeds were cultivated. FIG. 15 shows a PPES-treated synthetic seed containing viable plant material grown from axillary bud cells.

Here the liquid culture of Wasabia Japonica was undertaken with and without the addition of PPES to the culture medium. After a 4 week period, the Wasabia Japonica liquid cultures that contained 1% of a 250 mg/l solution of PPES, showed a mean 51% increase in optical density at 519 nm indicating increase cell proliferation after exposure to PPES. These cases show the potential of PPES as medium, adjuvant, growth stimulator, serum or other constituent in plant cell culture.

### Example 7

### Application of Chlorella Extract Purified According to this Invention (Chlorella Extract) to make Nutritional Products with a Range of Colors

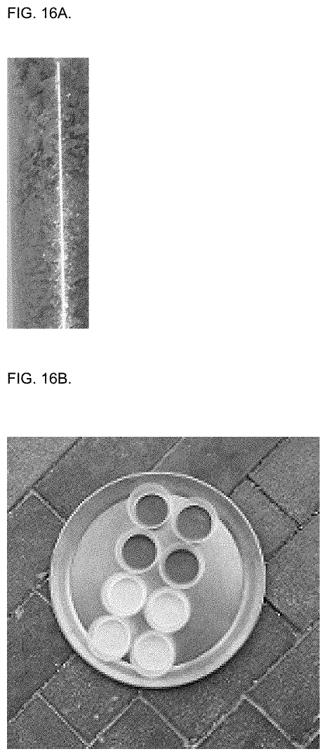

In this example, Chlorella extract is used to make a vegan mayonnaise. The Chlorella extract is slightly yellow-white or mustard in color, indicating that the purification process undertaken according to this invention has removed most of the chlorophyll, a source of a strong green color and unpleasant taste, which is predominantly present in Chlorella grown using light (as opposed to Chlorella grown under heterotrophic culture conditions such as Chlorella protothecoides). The vegan mayonnaise is made with vegetable oils, soy milk, lemon juice and the Chlorella extract through vigorous mixing or blending. FIG. 16A shows the Chlorella extract and FIG. 16B shows the vegan mayonnaise in one case colored with organic paprika powder and in the other case colored with Chlorella flour made with the dried and ground spent biomass from the purification process.

### Example 8

### Application of PC-Rich, Purified Extract of Spirulina Purified According to this Invention (PPES) to Accelerate and Improve Cell Culture of Mammalian Cells

Here, frozen Madin-Darby Canine Kidney (MDCK) cells at passage 30 were thawed and plated onto a treated T-25 flask. They were plated in growth media (GM) containing Dulbecco's modified eagles medium (DMEM) containing 4 mM L-glutamine, 4500 mg/L glucose, and 1 mM sodium pyruvate and supplemented with 10% fetal bovine serum. The cells were cultured in a carbon dioxide incubator as per standard mammalian cell culture conditions. The following day the MDCK cells were observed to be >90% confluent and subsequently passaged for 15 minutes using 0.04% Trypsin and 0.03% Ethylenediamine tetraacetic acid (EDTA). Cells were spun down at 300 g. Trypsin was deactivated using GM, and cells were passaged onto a treated t-75 flask at a subcultivation ratio of 1:2 as recommended by ATCC. Viability assay using trypsin blue dye indicated cell viability of >90%. The following day the MDCK cells were observed to be >90% confluent and subsequently passaged using the above protocol. Cell viability of >90% was observed, and cells were plated into two T-75 flasks. After 48 hours, cells were observed to be >90% confluent in T-75 flasks. 96 well plate was prepared for testing PPES. 50 μl of DMEM was added to 50 μl of experimental solution (DMEM, 250 mg/L PPES). Positive control wells received 100 μl of GM. Cells in T-75 flasks were passaged, counted, and plated into 96 well plates at a density of either 3e4 (Density 1) or 3e5 (Density 2). GM was used to deactivate trypsin, and 100 μl of cell solution were seeded onto each well with media that had been distributed as described above. Cells were grown for 24 hours. Post 24 hours, cell culture media was taken and replaced with 100 μl of same material, and 10 μl of 3-(4,5-dimethylthiazol-2-yl)-2,5-diphenyltetrazoliumbromide (tetrazolium dye). Cells were incubated for 3 hours. After 3 hours, 85 μl of media in each well was removed and 50 μl of dimethyl sulfoxide was added to solubilize formazan crystals that the mitochondria had metabolized from tetrazolium dye. Cells were incubated for 15 minutes. A Biotek ELX808IU Advanced Absorbance Microplate Reader was used to perform readings at a wavelength of 560 nm. Summary results omitting blanks are displayed below. The data shown below indicates that PPES increased metabolic output of MDCK cells, a finding indicative of PPES activity in enhancing proliferation in mammalian cell culture.

| | Density 1 | | Density 2 | |
|---|---|---|---|---|
| PPES | 0.099 | 0.104 | 0.025 | 0.026 |
| 5% FBS | 0.025 | 0.022 | 0.013 | 0.014 |
| 10% FBS | 0.044 | 0.036 | 0.018 | 0.014 |

### Example 9

### Application of PC-Rich, Purified Extract of Spirulina Purified According to this Invention (PPES) to Accelerate and Improve the Culture of Fungi Including Edible Mushrooms and Entheogenic or Medical Mushrooms in Various Culture Media

Here, Psilocybe, Malabar, Tampanensis, Koh Samui, P E, Gourmet edible, Chicken of the woods and Almond Agaricus were cultivated with and without PPES in generally accepted culture conditions and in liquid culture. In the case of Psilocybe liquid culture, it was found that the liquid cultures receiving PPES had an average 60% increase in optical density measured at 450 nm, indicative of an increase in cell density and hence increased proliferation following exposure to PPES.

It was observed that PPES accelerated fungal culture, with direct impact of growth velocity, time to maturity, growth cycle timing and biomass yield including the formation of hyphodial knots in liquid culture.

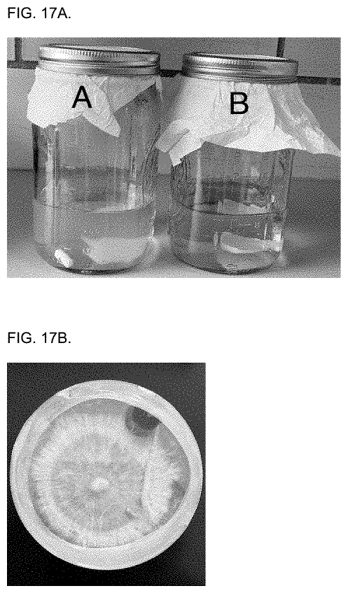

FIG. 17A shows the results after 24 hours of Psilocybe culture in 10 ml of Caro syrup in 300 ml of distilled water with (A)/without (B) addition of 0.03 g of PPES in a shaker incubator. FIG. 17B shows that a Malabar strain exposed to PPES in a petri dish that has completed its life cycle to spore release.

### Example 10

### Application of PC-Rich, Purified Extract of Spirulina Purified According to this Invention (PPES) as a Soil and Water Bioremediation Agent

A controlled, observational trial to test the bioremediation potential of PPES in hydrocarbon-polluted soil was conducted. The trial compared soil microbial activity, phenotype, biomass and photosynthetic parameters of corn and soy plants. It was demonstrated that periodic application of PPES increased bacterial activity and improved plant growth in biodiesel-polluted soil. PPES treated soil showed a 60% increase in soil microbial activity. Plants growing in PRSE treated soil had improved leaf length, stem diameter and total mass. PRSE treated plants showed a 20% increase in photosynthetic parameters (Performance Index). PPES has potential applications in the natural remediation of hydrocarbon-polluted soils.

| | PPES + | DIESEL |
|---|---|---|
| | DIESEL (PD) | (D) |
| Mean soy leaf | 74 | 60 |
| length (mm) | | |
| Mean corn stem | 94 | 88 |
| diameter (mm) | | |
| Total biomass (grams) | 1881 | 1640 |

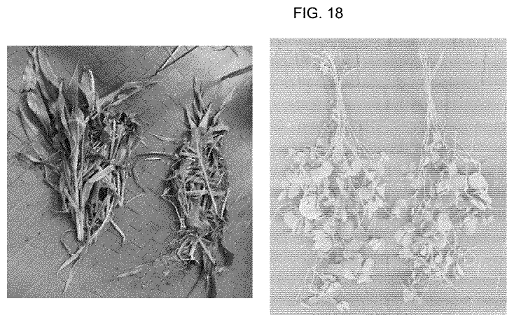

FIG. 18 illustrates the difference in biomass between the treated and untreated corn and soy groups.

Here also a controlled, observational trial to test the bioremediation potential of PPES in badly polluted river water (water from the South Branch of the Chicago River (“Bubbly Creek”) was conducted. It was demonstrated that PPES had a protective effect on lettuce grown under hydroponic conditions in the badly polluted river water.

### Example 11

### Application of Spent Material (By-Product) from which PCB Composition has Diffused as an Ingredient for Plant-Based Foods (Alternative, Non-Meat Protein)

The spent material (by-product) of PPES production can be dried and ground and used as a microalgae flour or alternative protein or plant-based food ingredient. This spent material has different (and improved) organoleptic, bacterial stability and shelf life properties as compared to dried Spirulina powder. Spent material (by-product) from Chlorella has similar properties. A recipe for a “plant-based burger” using the spent material (by-product) of PPES production is provided in FIG. 19.

1. A purified phycobiliprotein (PBP) composition characterized by one or more of the following characteristics:
a) the protein fraction of the composition comprises greater than about 30% of a protein having a molecular weight of about 17,695 kDa and an isoelectric point of about 6.29 as assayed by two dimensional gel electrophoresis followed by quantitation by densitometry;
b) the protein fraction of the composition comprises greater than about 5% of a protein having a molecular weight of about 19,833 kDa and an isoelectric point of about 6.14 as assayed by two dimensional gel electrophoresis followed by quantitation by densitometry;
c) the protein fraction of the composition comprises greater than about 1% of a protein having a molecular weight of about 45,578 kDa and an isoelectric point of about 6.2 as assayed by two dimensional gel electrophoresis followed by quantitation by densitometry;
d) the protein fraction of the composition comprises greater than about 1% of a protein having a molecular weight of about 35,014 kDa and an isoelectric point of about 5.9 as assayed by two dimensional gel electrophoresis followed by quantitation by densitometry;
e) the protein fraction of the composition comprises greater than about 0.30% of a protein having a molecular weight of about 24,688 kDa and an isoelectric point of about 5.3 as assayed by two dimensional gel electrophoresis followed by quantitation by densitometry;
f) the protein fraction of the composition comprises greater than about 2% of a protein having a molecular weight of about 22,522 kDa and an isoelectric point of about 5.9 as assayed by two dimensional gel electrophoresis followed by quantitation by densitometry;
g) the protein fraction of the composition comprises greater than about 1% of a protein having a molecular weight of about 21,023 kDa and an isoelectric point of about 7.3 as assayed by two dimensional gel electrophoresis followed by quantitation by densitometry;
h) the protein fraction of the composition comprises greater than about 0.50% of a protein having a molecular weight of about 13,417 kDa and an isoelectric point of about 7.3 as assayed by two dimensional gel electrophoresis followed by quantitation by densitometry;
i) the protein fraction of the composition comprises a ratio of major protein constituents to minor protein constituents of less than 3.5:1 based on the aggregate mass of the proteins, wherein major protein constituents are the protein having a molecular weight of about 17,695 kDa and an isoelectric point of about 6.29 as assayed by two dimensional gel electrophoresis followed by quantitation by densitometry and the protein having a molecular weight of about 19,833 kDa and an isoelectric point of about 6.14 as assayed by two dimensional gel electrophoresis followed by quantitation by densitometry and the minor protein constituents are the remainder of the proteins;
j) the protein fraction of the composition comprises less than about 75% on a mass basis of the combined amounts of the protein having a molecular weight of about 17,695 kDa and an isoelectric point of about 6.29 as assayed by two dimensional gel electrophoresis followed by quantitation by densitometry and the protein having a molecular weight of about 19,833 kDa and an isoelectric point of about 6.14 as assayed by two dimensional gel electrophoresis followed by quantitation by densitometry;
k) the composition produces a solution having a color value of greater than 200 E 10%/1cm when 250 mg of a dry powder of the composition are dissolved in one liter of water and absorbance is measured at 618 nm.

2. The purified phycobiliprotein composition of claim 1, wherein the composition has at least two of characteristics a, b, c, d, e, f, g, h, i, j and k.

3. The purified phycobiliprotein composition of claim 1, wherein the composition has at least three of characteristics a, b, c, d, e, f, g, h, i, j and k.

4. The purified phycobiliprotein composition of claim 1, wherein the composition has at least four of characteristics a, b, c, d, e, f, g, h, i, j and k.

5. The purified phycobiliprotein composition of claim 1, wherein the composition has at least five of characteristics a, b, c, d, e, f, g, h, i, j and k.

6. The purified phycobiliprotein composition of claim 1, wherein the composition has at least six of characteristics a, b, c, d, e, f, g, h, i, j and k.

7. The purified phycobiliprotein composition of claim 1, wherein the composition has at least seven of characteristics a, b, c, d, e, f, g, h, i, j and k.

8. The purified phycobiliprotein composition of claim 1, wherein the composition has at least eight of characteristics a, b, c, d, e, f, g, h, i, j and k.

9. The purified phycobiliprotein composition of claim 1, wherein the composition has at least nine of characteristics a, b, c, d, e, f, g, h, i, j and k.

10. The purified phycobiliprotein composition of claim 1, wherein the composition has at least ten of characteristics a, b, c, d, e, f, g, h, i, j and k.

11. The purified phycobiliprotein composition of claim 1, wherein the composition has all eleven characteristics a, b, c, d, e, f, g, h, i, j and k.

12. The purified PBP composition of claim 1, wherein the PBP composition is produced by a process comprising:
encapsulating Spirulina to provide capsules;
contacting the capsules with an aqueous medium under conditions such that the compound of interest passes from the capsule into the aqueous solution.

13. A PBP composition comprising a dried powder comprising a purified PBP composition according to claim 1, the powder having a residual moisture content of less than about 5% w/w of the powder.

14. A PBP composition according to claim 13, further comprising a second biologically active agent.

15. The PBP composition of claim 14, wherein the second biologically active agent is a pharmaceutical agent.

16. The PBP composition of claim 14, wherein the second biologically active agent is a nutraceutical agent.

17. An oral delivery vehicle comprising the PBP composition of claim 1.

18. The oral delivery vehicle of claim 17, wherein the oral delivery vehicle is selected from the group consisting of a capsule, a tablet and a gummi gel.

19. A formulation comprising a PBP composition according to claim 1 and a physiologically or pharmaceutically acceptable carrier.

20. A stabilized PBP composition comprising a phycobiliprotein composition according to claim 1 and a stabilizing agent and/or antioxidant that does not naturally occur in Arthrospira platensis.
